(19) United States
(12) Patent Application Publication
(10) Pub. No.: US 2011/0160682 A1
(43) Pub. Date: Jun. 30, 2011

(54) MEDICAL DEVICE

(76) Inventors: F. Nicholas Franano, Kansas City, MO (US); Daniel Philip Gottlieb, Sudbury, MA (US)

(21) Appl. No.: 12/865,379

(22) PCT Filed: Feb. 2, 2009
(86) PCT No.: PCT/US09/32891

§ 371 Date: Jan. 21, 2011

Related U.S. Application Data
(60) Provisional application No. 61/025,084, filed on Jan. 31, 2008.
(60) Provisional application No. 61/025,463, filed on Feb. 1, 2008.
(60) Provisional application No. 61/075,710, filed on Jun. 25, 2008.

(51) Int. Cl. A61M 5/32

(52) U.S. Cl. 604/272

(57) ABSTRACT

A medical device for the delivery of fluids into or through the wall of a biological space or conduit is described. The medical device comprises one or more actuators, which may exist in a constrained configuration which is substantially parallel to the longitudinal axis of the device or an unconstrained configuration in which at least a portion of the one or more actuators is oriented substantially non-parallel to the longitudinal axis of the device. The device further comprises tissue penetrators, which may be attached to the actuators or contained within the actuators. Upon positioning of the device at the target treatment site within a biological space or conduit, the operator may release the actuators from their constrained position, allowing the actuators to adopt their unconstrained position and make contact with an adjacent wall of a biological space or conduit. The actuators are configured to minimize the physical contact between the device and the target tissue, thereby permitting uniform and enhanced delivery of fluids into less compressed tissue, with reduced tissue trauma.

This application claims the priority benefit under 35 U.S.C. §119(e) of U.S. provisional application No. 61/025,084, filed Jan. 31, 2008; U.S. provisional application No. 61/025,463, filed Feb. 1, 2008; and U.S. provisional application No. 61/075,710, filed Jun. 25, 2008, the contents of each of which are incorporated by reference herein in their entireties.

### FIELD OF THE INVENTION

The present invention is generally related to medical devices, methods and kits for the delivery of fluids into or through a wall of a biological space or conduit and optionally into the tissue adjacent to a wall of the biological space or conduit. More preferably, the present invention is directed to medical devices and related methods and kits for the delivery of fluids into or through a wall of a biological space or conduit and optionally into the tissue within or adjacent to a wall of the biological space or conduit in a controlled, uniform and minimally disruptive manner.

### BACKGROUND

Numerous devices have been developed for the purpose of delivering fluids into and/or through blood vessel walls. For example, U.S. Pat. Nos. 5,873,852 and 6,210,392 describe a device that includes an inflatable balloon mounted on a catheter and a plurality of injectors that extend outwardly and are deployed in conjunction with inflation of the balloon. U.S. Pat. Nos. 5,873,852 and 6,210,392 also disclose devices in which a grommet is used in conjunction with push-pull wires to forcibly insert injectors of a fixed length into the vessel wall. Similarly, U.S. Pat. No. 6,638,246 discloses a catheter that utilizes a balloon comprising a plurality of microneedles mounted on its outer surface to deliver fluids into vessel walls. U.S. Pat. App. Publication No. 2006/0189941A1 discloses a catheter which utilizes numerous microneedles for distributing fluids into the adventitial tissue of a blood vessel. Each of these publications is incorporated herein by reference in its entirety.

Drug delivery catheters with needles whose penetration depths into surrounding target tissues can be modulated also have been disclosed. For example, U.S. Patent No. 5,354,279 discloses a catheter with a plurality of needles that can be extended (in unison) from the catheter head and simultaneously deflected forward and laterally for penetration to varying depths into the wall of a blood vessel. Similarly, U.S. Pat. No. 7,141,041 discloses a catheter with a single needle that can be simultaneously advanced along the longitudinal axis of the catheter and deflected perpendicularly to the longitudinal axis of the catheter for penetration into the tissues surrounding blood vessels or other body lumens.

In spite of these and other disclosures of devices for delivery of fluids to walls of biological spaces or conduits, devices that provide for more uniform, consistent and less disruptive and traumatic delivery of fluids into or through the wall of a biological space or conduit while allowing for penetration of the tissue penetrator(s) to a desired depth are still needed.

### SUMMARY OF THE INVENTION

The present invention provides a medical device for insertion into a biological space or conduit, methods for using the medical device, and kits comprising the medical device.

In certain aspects, the present invention provides a medical device comprising at least one actuator having a constrained configuration, in which the at least one actuator is oriented substantially parallel to the longitudinal axis of the medical device, and an unconstrained configuration, in which at least a portion of the at least one actuator is oriented substantially non-parallel to the longitudinal axis of the medical device, and in which the at least one actuator, upon the removal of a constraining force, adopts the unconstrained configuration without the necessity for the external application of a deforming force. In a preferred embodiment, the unconstrained configuration of the at least one actuator has a predetermined shape. In another preferred embodiment, the unconstrained configuration is dimensioned to make contact with the inner surface of the wall of the biological space or conduit into which the medical device has been inserted. In yet another preferred embodiment, the transition from the constrained configuration to the unconstrained configuration occurs at a rate that is dependent, after removal of the constraining force, upon the physical properties of the resilient material from which the at least one actuator is constructed rather than at a rate that is dependent upon the input of external physical force by an operator.

The present invention further provides a method for delivering a fluid into or through a wall of a biological conduit, the method comprising the steps of introducing the medical device of the present invention into the biological conduit, advancing the device to a target site within the biological conduit, releasing the at least one actuator from a constrained configuration, and delivering at least one fluid into or through a wall of the biological conduit, thereby delivering a fluid into or through the wall of the biological conduit for therapeutic, prophylactic, diagnostic or other uses. In a preferred embodiment, the method further comprises the steps of returning the at least one actuator to a constrained configuration for repositioning of the device within the same biological conduit for delivering additional fluid, for repositioning of the device in a different biological conduit for delivering additional fluid, or for removing the device from said biological conduit. In another preferred embodiment, the biological conduit is a blood vessel. In yet another preferred embodiment, the fluid to be delivered by the medical device comprises an elastase.

In yet another aspect, the present invention provides a kit comprising a medical device of the present invention and at least one therapeutic agent or at least one diagnostic agent. In a preferred embodiment, the kit comprises an elastase.

The present invention advantageously permits precise placement of needles or other similar tissue penetrators into the target delivery site in or through the wall of a biological space or conduit. The precise placement of the tissue penetrators of the device of the present invention may be achieved through a conformational change of one or more actuators to which the tissue penetrators are attached or within which the tissue penetrators are otherwise contained. Preferably, this change occurs upon removal by the operator of the constraining force and without the input by the operator of any deforming forces to the device or the target tissue. This latter feature of the device permits the reproducible application of a known, predetermined and consistent force to the wall during treatment. Advantageously, the precise placement of the tissue penetrators achieved by the present invention may minimize the amount of physical contact between the device and the wall, thereby avoiding undue compression of the wall. This feature limits the trauma to the treatment site and enhances delivery of fluids into the less compressed and traumatized tissue. The present invention also provides a medical device capable of distributing fluids through a plurality of tissue penetrators in a uniform manner, whereby similar amounts of fluid are delivered through each tissue penetrator. The present invention advantageously permits user-controlled distribution of different amounts or types of fluid through each individual tissue penetrator if so desired. In a preferred embodiment, the present invention permits repositioning or removal of the medical device such that no portion of the device remains at or in the target site after administration of the fluid.

The medical device of the present invention also advantageously permits modulation of the depth to which a needle or other similar tissue penetrator penetrates into a target layer of a wall of a biological space or conduit or the tissue beyond a wall of a biological space or conduit, and advantageously provides for independent control of the desired penetration depth of each of a plurality of tissue penetrators. Moreover, the present invention allows for the use of tissue penetrators with diameters much smaller than conventional needles, if desired, because the medical device of the present invention, in certain specific embodiments, effects wall contact of a biological space or conduit with a larger diameter actuator through which a smaller diameter tissue penetrator may be advanced into or through the wall of the biological space or conduit.

### BRIEF DESCRIPTION OF THE DRAWINGS

FIG. 1 is a side, partially sectioned view of one embodiment of the medical device of the present invention.

FIG. 2 is an end section view in the plane of line 2-2 in FIG. 1.

FIG. 3 is an end section view in the plane of line 3-3 in FIG. 1.

FIG. 4 is a view similar to FIG. 1 that illustrates the movement of the actuators of the medical device.

FIG. 5 is a view of the medical device shown in FIG. 1, but with the central catheter component rotated 90° relative to its orientation in FIG. 1.

FIG. 6 is a partial view of the exterior of the medical device of FIG. 1 in its constrained position.

FIG. 7 is a diagram of the fluid path of the medical device of FIG. 1, extending from the Luer hubs through the fluid delivery conduits to the reservoir and then to the tissue penetrators.

FIG. 8 is a side, partially sectioned view of a second embodiment of the medical device of the present invention showing the actuators in their constrained configurations.

FIG. 9 is a view similar to FIG. 8, but showing the actuators in their unconstrained configurations.

FIG. 10 is an end perspective view of the assembly along the line 3′-3′ of FIG. 8.

FIG. 11 is an end perspective view of the assembly along the line 4′-4′ of FIG. 10 showing the tissue penetrators.

FIG. 12 is a side view showing the detail of the proximal end of the device, shown to the right in FIGS. 8 and 9.

FIG. 13 is a three-dimensional depiction of one embodiment of a medical device of the invention in its deployed, or unconstrained, configuration.

FIG. 14 shows the device of FIG. 13 in its undeployed, or constrained, configuration (top panel) as well as top (middle panel) and side (bottom panel) views of the device in its deployed configuration.

FIG. 15 shows a close up of one injection unit in a device of FIG. 13 (left panel) in the undeployed configuration, and a cross section view along the needles of the injection unit.

FIGS. 16A-16D depict the device of FIG. 13 as it is deployed. FIG. 16A shows the undeployed configuration; FIGS. 16B and 16C depict the device in a partially deployed configuration, and FIG. 16D shows the device in the fully deployed configuration.

FIG. 17 is a three-dimensional depiction of one embodiment of a medical device of the invention in its deployed, or unconstrained, configuration.

FIGS. 18A-18C depict the device of FIG. 17 as it is deployed. FIG. 18A shows the undeployed configuration; FIG. 18B depicts the device in a partially deployed configuration, and FIG. 18C shows the device in the fully deployed configuration.

FIG. 19 is a depiction of one embodiment of a medical device of the invention in its deployed, or unconstrained, configuration.

FIGS. 20A-20B. FIG. 20A is a close up depicting the needle configuration in the device. FIG. 20B shows a close up of the central catheter component and the proximal portion of the splines and the proximal needles (left panel) and a cross section view along the proximal needles (right panel).

FIGS. 21A-21D depict the device of FIG. 19 as it is deployed. FIG. 21A shows the undeployed configuration; FIGS. 21B and 21C depict the device in a partially deployed configuration, and FIG. 21D shows the device in the fully deployed configuration.

FIGS. 22A-E are photographs of a prototype of one embodiment of a medical device of the invention as it is deployed. FIG. 22A shows the undeployed configuration; FIGS. 22B, 22C and 22D depict the device in a partially deployed configuration, and FIG. 22E shows the device in the fully deployed configuration.

### DETAILED DESCRIPTION OF THE INVENTION

As used herein, a “wall” is any surface of any biological space or conduit, e.g., an inner or outer wall of a biological conduit such as a blood vessel. Examples of biological spaces include, but are not limited to, the peritoneal cavity, the epidural space, the arachnoid and subarachnoid spaces, the subdural space, or any potential spaces that may be created by separating two adjacent bodily tissues. A “biological conduit” is any tubular structure that conveys any fluid, gas, solid, colloid, or combination thereof from one location to another within an organism. In a preferred embodiment, the organism is a mammal, most preferably a human. Examples of biological conduits include, but are not limited to, arteries, veins, ureters, bronchi, bile ducts, glandular ducts, pancreatic ducts, urogenital conduits and gastrointestinal conduits.

In one embodiment, the medical device of the present invention has a central longitudinal axis, and comprises one or more actuators, wherein the one or more actuators can exist in a constrained configuration in which a length of said one or more actuators is oriented substantially parallel to the longitudinal axis of said medical device and an unconstrained configuration in which at least a portion of the length of said one or more actuators is oriented substantially non-parallel to the device's central longitudinal axis. After the device is positioned at a target site adjacent to the wall of a biological space or conduit, one or more actuators (and if desired, all of the actuators) may be released from a constrained configuration and permitted to adopt an unconstrained configuration, thereby making contact with the wall of the biological space or conduit. The one or more actuators may be of any shape, and in preferred embodiments, the movement of the one or more actuators from the constrained configuration to the unconstrained configuration occurs upon release of a constraining force by the device operator but without the input by the operator of any deforming forces to the device or the target tissue.

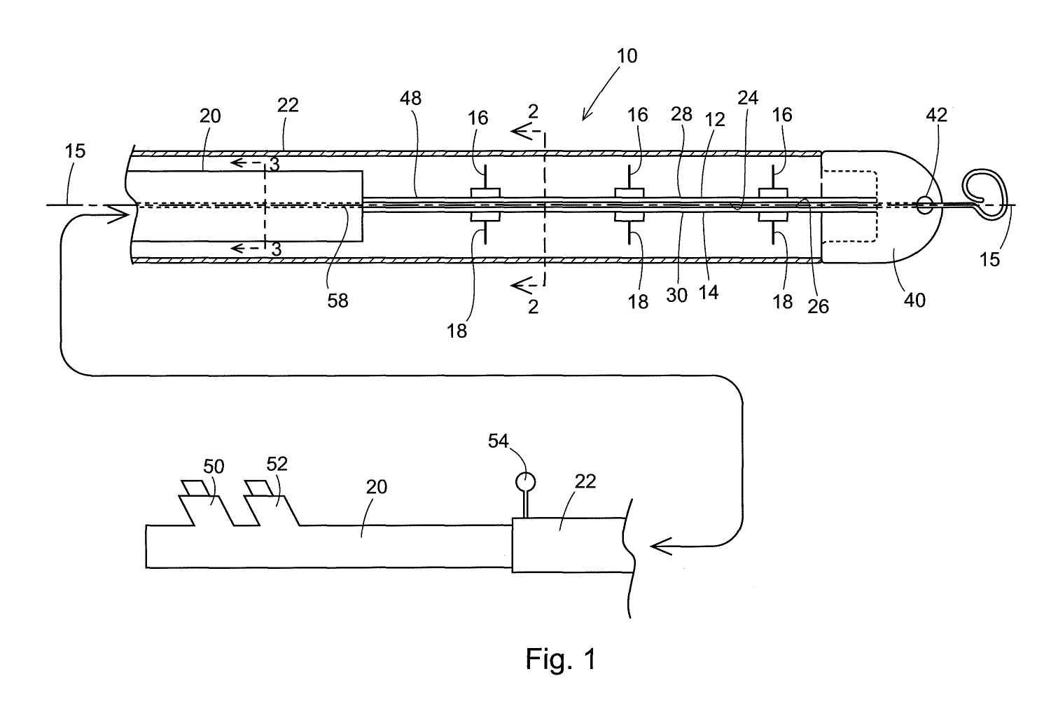

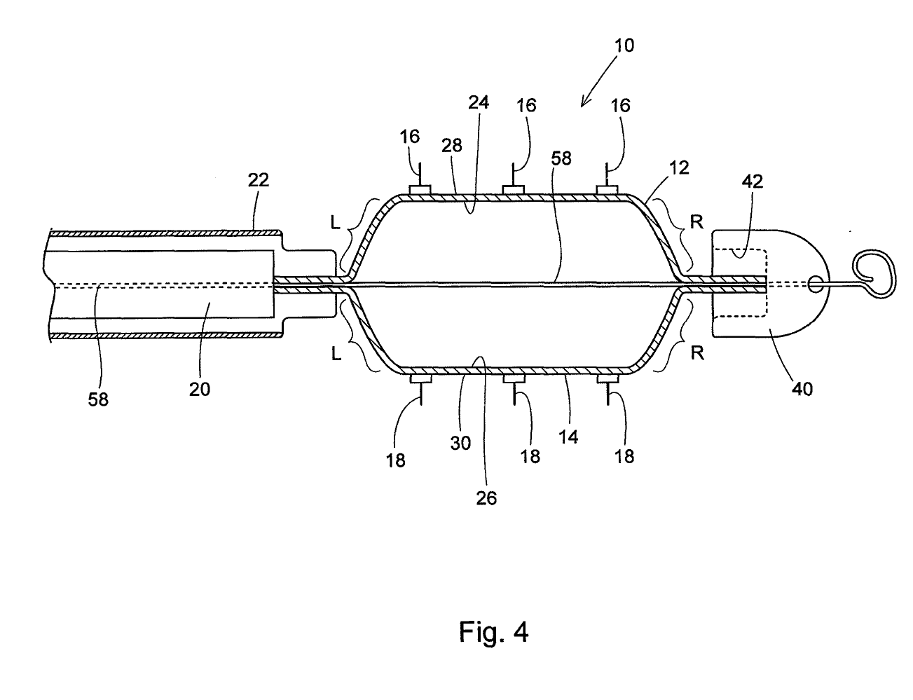

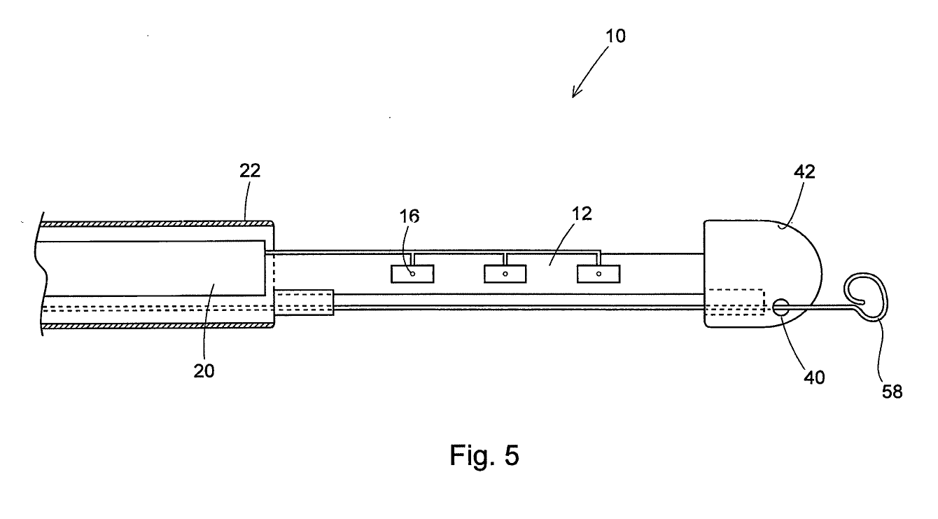

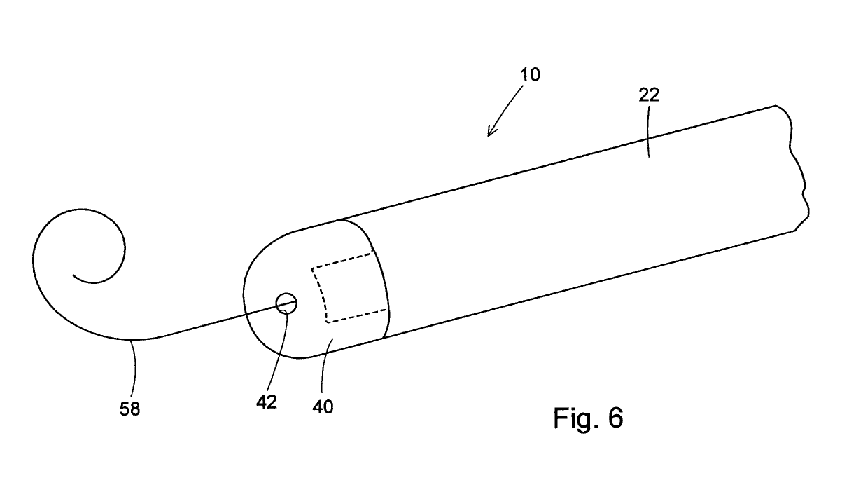

In a first specific embodiment, shown in FIG. 1, a device of the present invention is a fluid delivery catheter 10 comprising one or more actuators that are formed as a pair of elongate splines 12, 14, the intermediate regions of which are movable between a constrained configuration which is oriented substantially parallel to the central longitudinal axis 15 of the catheter assembly and an unconstrained configuration in which at least a portion of the pair of splines is oriented substantially non-parallel to said central longitudinal axis (see the left L and right R portions of the spline lengths in FIG. 4). The one or more splines 12, 14 may be constructed as elongate bands or wires that each have opposite proximal and distal ends. In a preferred embodiment, the splines have flat, opposing interior surfaces 24, 26, and flat opposite facing exterior surfaces 28, 30. In this embodiment, the splines 12, 14 can translate between constrained positions and unconstrained positions, as shown respectively in FIGS. 1 and 4. In one embodiment, the pair of splines is positioned back-to-back in their constrained configurations as shown in FIG. 1.

The catheter 10 further comprises one or more tissue penetrators 16, 18 secured to one or more surfaces of the one or more splines 12, 14, a central catheter component 20 having an elongate length, and an exterior catheter component 22 (sometimes referred to herein as a sheath) that can shield the tissue penetrator or penetrators during catheter movement within the biological space or conduit.

The tissue penetrators 16, 18 may be constructed of any suitable material. Preferred examples of such materials include, but are not limited to, nickel, aluminum, steel and alloys thereof. In a specific embodiment, the tissue penetrators are constructed of nitinol.

The central catheter component 20 and the exterior catheter component 22 may be constructed of materials typically employed in constructing catheters. Examples of such materials include, but are not limited to, silicone, polyurethane, nylon, Dacron, and PEBAX™.

The actuators are preferably constructed of a flexible, resilient material. In a preferred embodiment, the flexible, resilient material is capable of being constrained upon the application of a constraining force, e.g., when the actuators are in the constrained configuration, and adopts its original unconstrained shape when the constraining force is removed, e.g., when the actuators are in the unconstrained configuration. Any such flexible, resilient material can be used, including but not limited to surgical steel, aluminum, polypropylene, olefinic materials, polyurethane and other synthetic rubber or plastic materials. The one or more actuators are most preferably constructed of a shape memory material. Examples of such shape memory materials include, but are not limited to, copper-zinc-aluminum-nickel alloys, copper-aluminum-nickel alloys, and nickel-titanium (NiTi) alloys. In a preferred embodiment, the shape memory material is nitinol. In a preferred embodiment, when the pair of splines assumes the unconstrained configuration, the shape memory properties of the material from which each spline is formed cause the splines, without the application of any external deforming force, to bow radially away from each other in a single plane as shown in FIG. 4.

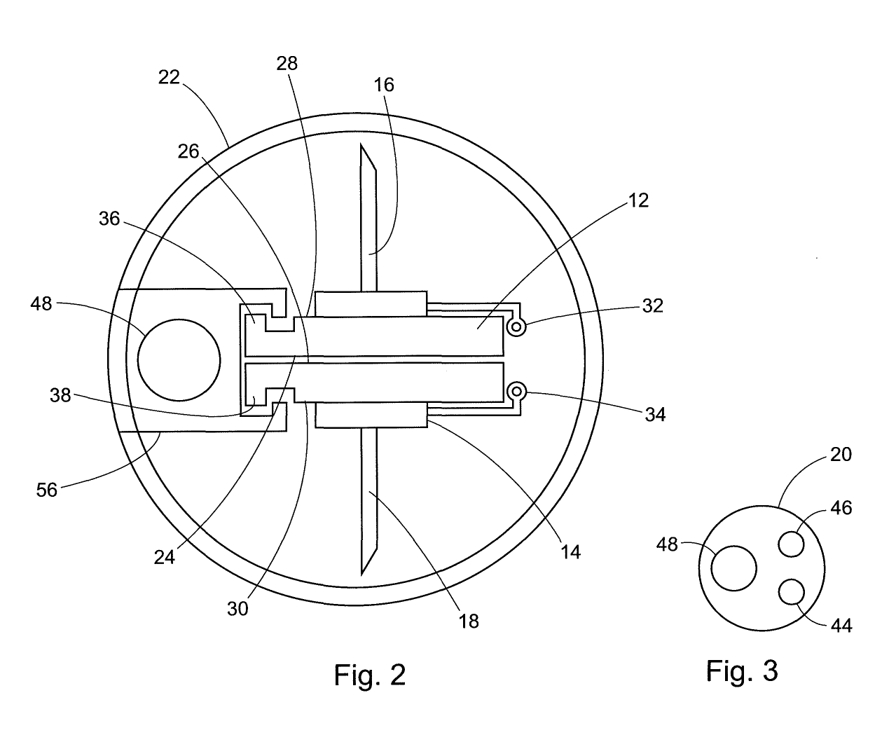

One or more of the splines (and preferably each of the splines) has a flexible fluid delivery conduit 32, 34 that extends along the length of the spline, or within the spline, as shown in FIG. 2. As the splines 12, 14 move from their straight, constrained configurations to their bowed, unconstrained configurations, the fluid delivery conduits 32, 34 also move from straight configurations to bowed configurations. In one embodiment, the fluid delivery conduits 32, 34 are separate tubular conduits that are secured along the lengths of the pair of splines 12, 14. In another embodiment, the fluid delivery conduits are conduits formed into or within the material of the splines.

One or more of the splines (and preferably each of the splines 12,14) is also formed with a zipper rail 36, 38 that extends along a length of the spline (FIG. 2). The zipper rails 36, 38 are formed of either the same material as the splines 12, 14, or a material that flexes with the splines 12, 14.

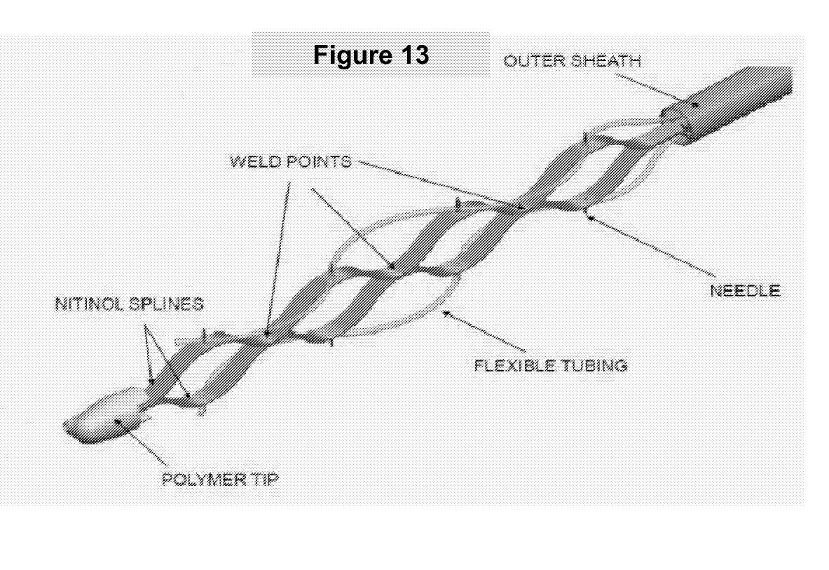

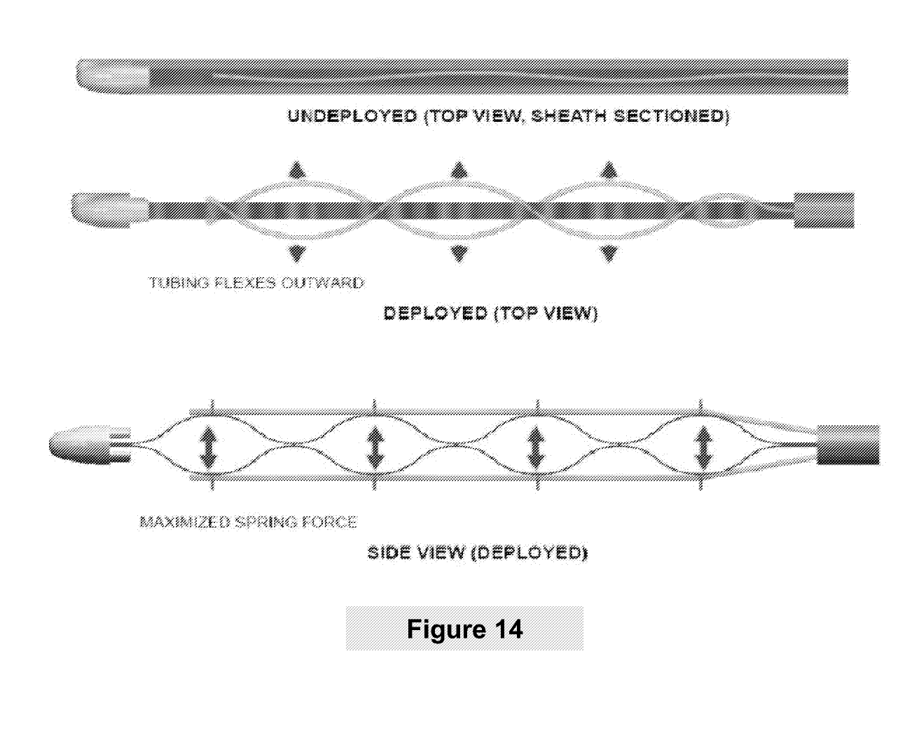

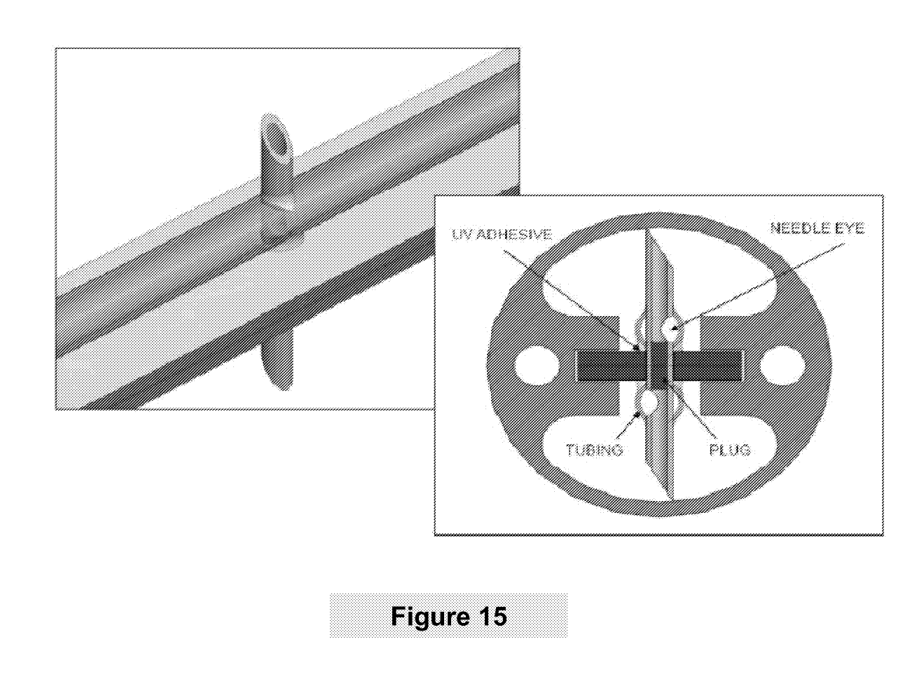

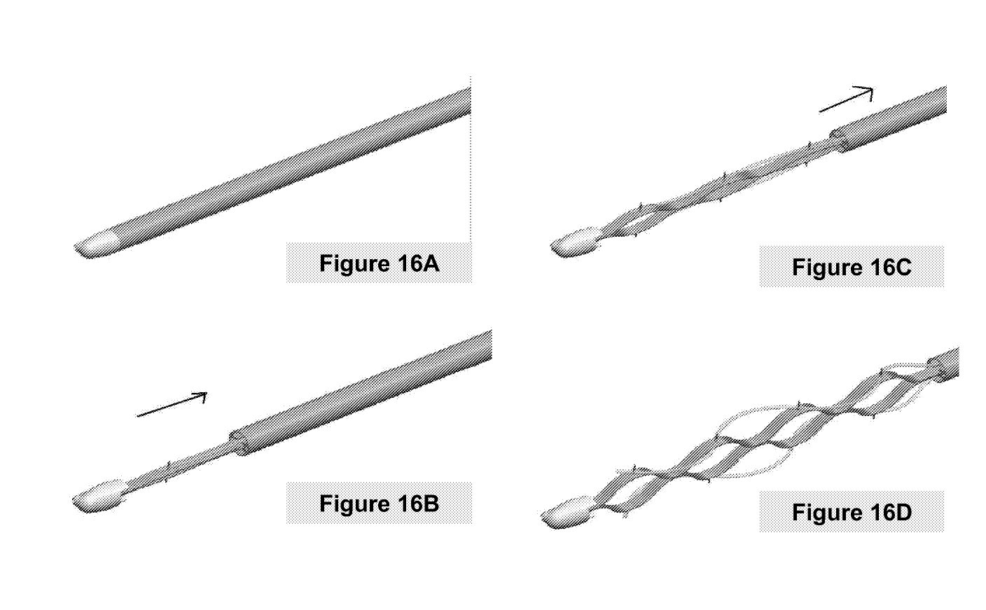

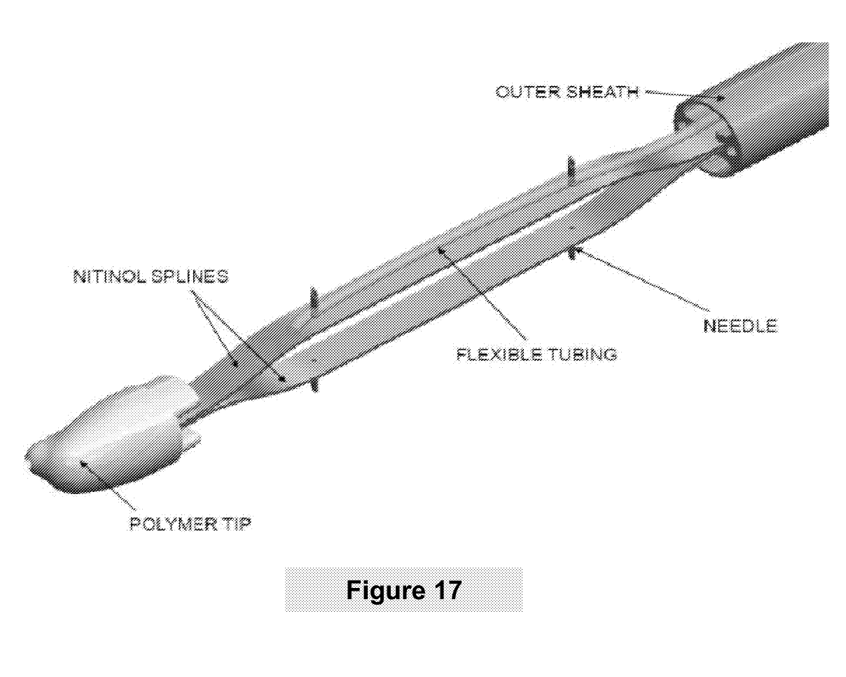

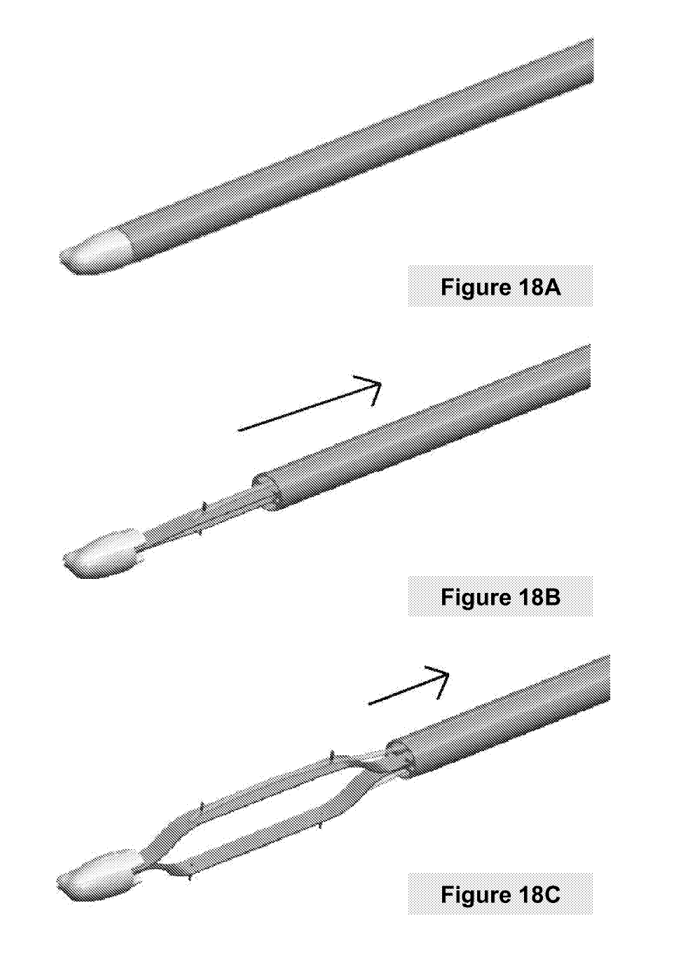

In certain aspects, a medical device of the invention comprises a pair of splines that are attached, e.g., by welding, at certain intervals along their lengths, as depicted in FIGS. 13 to 16. In this configuration, each portion of the spline between the two attachments, referred to herein as an “injection unit,” moves from a straight configuration to a bowed configuration independently of other portions of the spline, or other injection units. See, e.g., FIG. 16C showing different injection units at different degrees of constraint. The use of splines with multiple injection units minimizes wall contact. Each injection unit preferably has at least one pair of opposing tissue penetrators, such that at least one tissue penetrator is secured to the surface of the portion of each spline within the injection unit (i.e., one above the central longitudinal axis and one below the central longitudinal axis), although it is contemplated that an injection unit can have more than one tissue penetrator attached to each spline portion within it. In certain embodiments, a device of the invention has a single injection unit, two injection units, three injection units, four injection units, five injection units or six injection units.

One or more of the tissue penetrators 16, 18 is secured to the exterior surfaces 28, 30 of the pair of splines 12, 14 (FIG. 2). The tissue penetrators 16, 18 are connected to and communicate with the fluid delivery conduits 32, 34 that extend along the lengths of the splines 12, 14. The tissue penetrators 16, 18 are positioned to project substantially perpendicular from the exterior surfaces 28, 30 of the splines 12, 14. The tissue penetrators 16, 18 have hollow interior bores that communicate with the fluid delivery conduits 32, 34 of the splines. The distal ends of the tissue penetrators have fluid delivery ports that communicate with the interior bores of the tissue penetrators.

The device permits delivery of fluids into or through one or more distinct layers of a wall of a biological conduit or space, for example a vascular wall. The vascular wall comprises numerous structures and layers, including the endothelial layer and basement membrane layer (collectively the intimal layer), the internal elastic lamina, the medial layer, and the adventitial layer. These layers are arranged such that the endothelium is exposed to the lumen of the vessel and the basement membrane, the internal elastic lamina, the media, and the adventitia are each successively layered over the endothelium, as described in U.S. Pat. App. Publication No. 2006/0189941A1. With the medical devices of the present invention, the depth to which the tissue penetrators 16, 18 can penetrate is determined by the length of each tissue penetrator 16, 18. For example, if the target layer is the adventitial layer, tissue penetrators 16, 18 having a defined length sufficient for penetration to the depth of the adventitial layer upon deployment of the device are used. Likewise, if the target layer is the medial layer, tissue penetrators 16, 18 having a defined length sufficient for penetration to the depth of the medial layer upon deployment of the device are used.

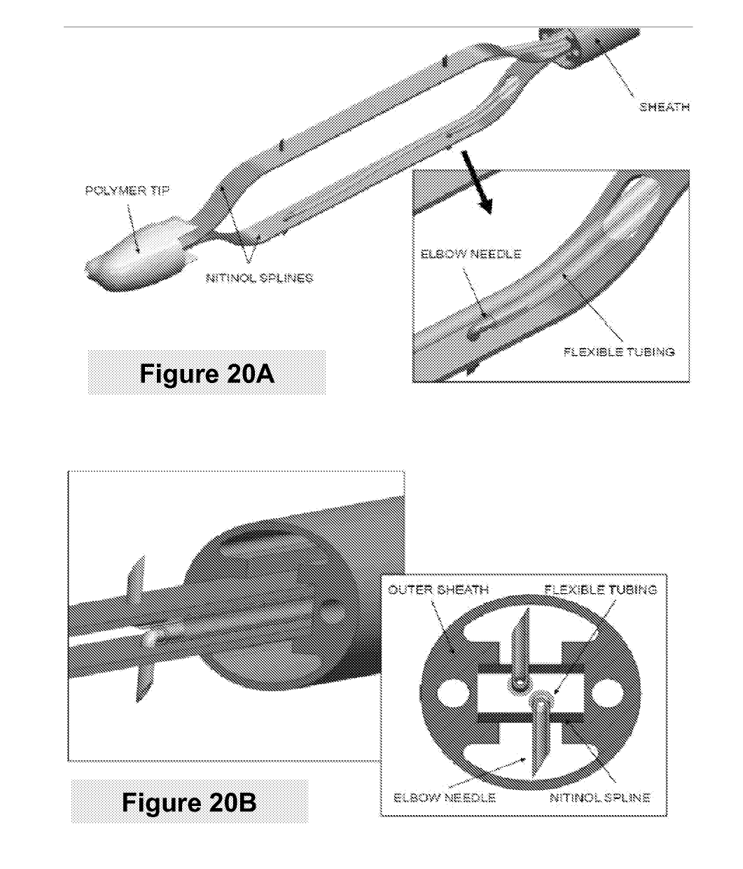

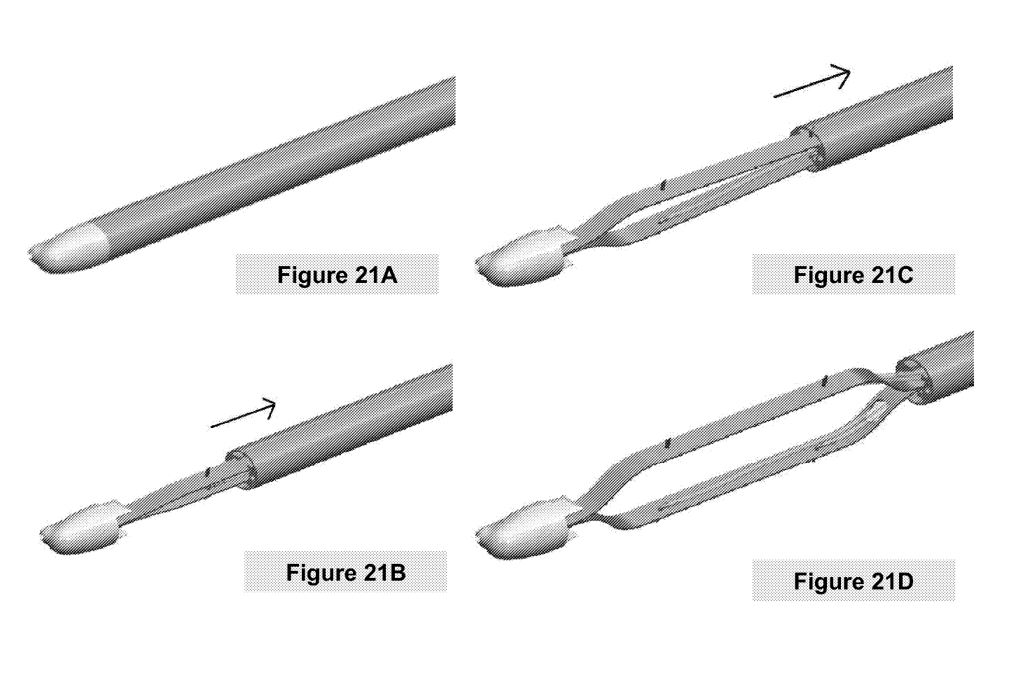

In specific embodiments, the length of tissue penetrators 16, 18 may range from about 0.3 mm to about 5 mm for vascular applications, or up to about 20 mm or even 30 mm for applications involving other biological spaces or conduits, for example in colonic applications. Tissue penetrators 16, 18 preferably have a diameter of about 0.2 mm (33 gauge) to about 3.4 mm (10 gauge), more preferably 0.2 mm to 1.3 mm (about 33 to 21 gauge). The distal tips of the tissue penetrators may have a standard bevel, a short bevel, or a true short bevel. In an alternative embodiment, the tissue penetrators attached to any one spline are not of identical lengths, but may be configured such that their distal ends align so as to be equidistant from the wall of the biological space or conduit when the medical device is in the unconstrained position, e.g., during use. In certain embodiments, tissue penetrators are attached, e.g., soldered or glued, to the splines, as shown in the embodiment of FIG. 17. In other embodiments, the tissue penetrators are elbow needles presented at the surface of the splines through a hole in the splines, as shown in the embodiment of FIG. 20.

The central catheter component 20 has an elongate length with opposite proximal and distal ends, shown to the left and right respectively in FIG. 1. In one embodiment, the central catheter component 20 has a cylindrical exterior surface that extends along its elongate length. The proximal ends of the splines 12, 14 are attached e.g., soldered or glued, to the distal end of the central catheter component 20, while the distal ends of the splines 12, 14 are attached, e.g., soldered or glued, to a catheter guide tip 40. The tip 40 has a smooth exterior surface that is designed to move easily in the biological conduit. A guide wire bore 48 extends through the length of the central catheter 20 and tip 40. The guide wire bore is dimensioned to receive a guide wire in sliding engagement through the bore.

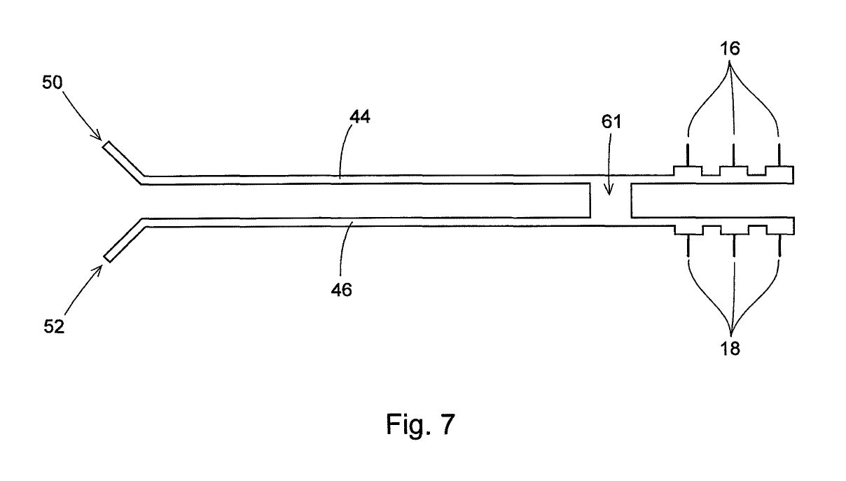

A pair of fluid delivery lumens 44, 46 extends through the interior of the central catheter component 20 for the entire length of the catheter component (FIG. 3). At the distal end of the central catheter component 20 the pair of fluid delivery lumens 44, 46 communicates with the pair of fluid delivery conduits 32, 34 that extend along the lengths of the splines 12, 14 to the tissue penetrators 16, 18. A guide wire bore 48 also extends through the interior of the central catheter component 20 from the proximal end to the distal end of the central catheter component (FIG. 3). The proximal end of the central catheter component 20 is provided with a pair of Luer hubs 50, 52 (FIG. 1). In one embodiment, each Luer hub 50, 52 communicates with one of the fluid delivery lumens 44, 46 extending through the length of the central catheter. Each Luer hub 50, 52 is designed to be connected with a fluid delivery source to communicate a fluid through each Luer hub 50, 52, then through each fluid delivery lumen 44, 46 extending through the central catheter component 20, then through each fluid delivery conduit 32, 34 extending along the lengths of the pair of splines 12, 14, and then through the tissue penetrators 16, 18 secured to each of the pair of splines. In another embodiment, each Luer hub 50, 52 independently communicates with both of the fluid delivery lumens 44, 46 extending through the length of the central catheter component. In this configuration, a first fluid can be delivered through a first Luer hub to both tissue penetrators 16, 18 and a second fluid can be delivered through a second Luer hub to both tissue penetrators 16, 18. Delivery of fluid to both tissue penetrators from each Luer hub can be achieved by an independent conduit extending from each Luer hub to a distal common reservoir 61 as shown in FIG. 7. This reservoir communicates with both tissue penetrators 16, 18. Alternatively, in another embodiment, the medical device of the instant invention comprises only a single Luer hub connected to a single fluid delivery lumen extending through the central catheter, which then is attached to a distal common reservoir, permitting the delivery of a single fluid to both tissue penetrators 16, 18.

The exterior catheter component 22 has a tubular configuration that surrounds the pair of splines 12, 14 and a majority of the central catheter 20 (FIG. 1). The catheter component 22 has an elongate length that extends between opposite proximal and distal ends of the catheter component shown to the left and right, respectively in FIG. 1. The catheter component distal end is dimensioned to engage in a secure engagement with the guide tip 40, where the exterior surface of the tip 40 merges with the exterior surface of the catheter component 22 when the catheter component distal end is engaged with the tip. The tubular configuration of the catheter component 22 is dimensioned so that an interior surface of the catheter component 22 is spaced outwardly of the plurality of tissue penetrators 16, 18 on the pair of splines 12, 14 in the constrained positions of the pair of splines. The proximal end of the central catheter 20 extends beyond the proximal end of the catheter component 22 when the catheter component distal end engages with the catheter guide tip 40.

A mechanical connection 54 is provided between the exterior catheter component 22 proximal end and the central catheter component 20 proximal end that enables the exterior catheter component to be moved rearwardly along the lengths of the pair of splines 12, 14 and the central catheter component 20 causing the exterior catheter component 22 distal end to separate from the guide tip 40 and pass over the pair of splines 12, 14, and forwardly over the length of the central catheter component 20 and over the lengths of the pair of splines 12, 14 to engage the exterior catheter component 22 distal end with the tip 40 (FIG. 1). The mechanical connection 54 could be provided by a handle or button that manually slides the exterior catheter component 22 over the central catheter component 20. The connection 54 could also be provided by a thumbwheel or trigger mechanism. In addition, the connection 54 could be provided with an audible or tactile indicator (such as clicking) of the incremental movement of the exterior catheter component 22 relative to the central catheter component 20.

In one embodiment, the exterior catheter component 22 is provided with a single zipper track 56 that extends along the entire length of one side of the exterior catheter component 22 on the interior surface of the exterior catheter component (FIG. 2). The zipper track 56 in the interior of the exterior catheter component 22 engages in a sliding engagement with the zipper rails 36, 38 at one side of each of the splines 12, 14. Advancing the exterior catheter component 22 forwardly along the lengths of the central catheter component 20 and the pair of splines 12, 14 toward the guide tip 40 of the catheter assembly causes the zipper track 56 of the exterior catheter component to slide along the rails 36, 38 of the pair of splines 12, 14. This moves the pair of splines 12, 14 from their bowed, unconstrained configuration shown in FIG. 4 toward their back-to-back, constrained configuration shown in FIG. 1. The engagement of the spline rails 36, 38 in the zipper track 56 of the exterior catheter component 22 holds the pair of splines 12, 14 in their back-to-back relative positions shown in FIG. 1. With the exterior catheter component 22 pushed forward over the central catheter component 20 and the pair of splines 12, 14 to where the distal end of the exterior catheter component 22 engages with the guide tip 40, the tissue penetrators 16, 18 are covered and the catheter assembly of the present invention can be safely moved forward or backward in a biological space or conduit. The exterior catheter component 22 covers the tissue penetrators 16, 18 projecting from the pair of splines 12, 14 and the engagement of the exterior catheter component 22 with the distal guide tip 40 provides the catheter assembly with a smooth exterior surface that facilitates the insertion of the catheter assembly into and through a biological space or conduit such as a blood vessel. In another embodiment, the exterior catheter component 22 is provided with two zipper tracks at 180 degrees from each other that extend along the entire length of the exterior catheter component 22 on the interior surface and the splines have rails on both sides.

A guide wire 58 is used with the catheter assembly (FIG. 1). The guide wire 58 extends through the central catheter component guide wire bore 48, along the splines 12, 14, and through the guide tip outlet 42. In certain embodiments, the guide wire 58 has a solid core, e.g., stainless steel or superelastic nitinol. The guide wire may be constructed of radiopaque material, either in its entirety or at its distal portions (e.g., the most distal 1 mm to 25 mm or the most distal 3 mm to 10 mm). The guide wire 58 may optionally be coated with a medically inert coating such as TEFLON®.

In use of this device, the guide wire 58 is positioned in the biological space or conduit by methods well known in the art. The guide wire 58 extends from the biological space or conduit, through the guide wire outlet 42 in the tip 40 of the assembly, through the exterior shielding catheter 22 past the tissue penetrators 16, 18, and through the guide wire bore 48 of the central catheter 20. In other embodiments, the catheter assembly is a rapid-exchange catheter assembly, wherein the guide wire lumen is present in the distal end of the guide tip 40 of the catheter, but does not extend throughout the entire length of the medical device.

After positioning of the guide wire, the device is advanced into the biological space or conduit along the previously positioned guide wire 58. One or more radiopaque markers may optionally be provided on the device to monitor the position of the device in the biological space or conduit. Any material that prevents passage of electromagnetic radiation is considered radiopaque and could be used. Preferred radiopaque materials include, but are not limited to, platinum, gold, or silver. The radiopaque material can be coated on the surface of all or a part of the tip 40, on all or part of the splines 12, 14 or other actuators, on the guide wire 58, or on some combination of the foregoing strucutres. Alternatively, a ring of radiopaque material can be attached to the tip 40. The device may optionally be provided with onboard imaging, such as intravascular ultrasound or optical coherence tomography. The tip of the device may optionally be provided with optics that are used to determine the position of the device or characteristics of the surrounding biological space or conduit.

When the device is at its desired position in the biological space or conduit, the operator uses mechanical connection 54 to retract the exterior catheter component 22 rearwardly away from the guide tip 40. In a preferred embodiment, as the exterior catheter component 22 is withdrawn from over the tissue penetrators 16, 18, the zipper track 56 of the exterior catheter component 22 is withdrawn over the rails 36, 38 of the pair of splines 12, 14. This movement releases the pair of splines 12, 14 from their constrained, back-to-back configuration shown in FIG. 1, and allows the shape memory material of the splines 12, 14 to adopt their unconstrained, bowed configurations shown in FIG. 4. As the splines 12, 14 move to their unconstrained, bowed configurations, the splines come into contact with the inner surface of the wall(s) of the biological space or conduit and the tissue penetrators 16, 18 on the exterior surfaces 28, 30 of the splines 12, 14 are pressed into the interior surface of the biological space or conduit at the position of the device.

After the tissue penetrators 16, 18 have entered the desired layer of the wall of a biological space or conduit, a fluid can be delivered through the fluid delivery lumens 44, 46 in the central catheter component 20, through the fluid delivery conduits 32, 34 on the pair of splines 12, 14, and through the tissue penetrators 16, 18. When the delivery of the fluid is complete, the operator uses the mechanical connection 54 to move the exterior catheter component 22 (which may also be referred to as a shielding component) forward over the central catheter component 20 and over the pair of splines 12, 14 toward the guide tip 40. As the exterior catheter component 22 moves forward over the pair of splines 12, 14, the zipper track 56 on the interior of the exterior catheter component 22 passes over the rails 36, 38 on the pair of splines 12, 14, causing the splines 12, 14 to move from their unconstrained, bowed configuration back to their constrained configuration. When the exterior catheter component 22 has been entirely advanced over the pair splines 12, 14 and again engages with the guide tip 40, the zipper track 56 in the exterior catheter component 22 holds the splines 12, 14 in their constrained configuration. The device then can be repositioned for release at another location in the biological space or conduit or another biological space or conduit, or withdrawn from the body.

The shape and length of the splines 12, 14 are selected such that various embodiments of the device can be used in biological spaces or conduits of various sizes or diameters. In certain embodiments, the splines may be flat or rounded. Flat splines preferably have a width ranging from about 0.2 mm to about 20 mm, a height ranging from about 0.2 mm to about 5 mm, and a length ranging from about 10 mm to about 200 mm, depending on the particular application. Rounded splines preferably have a diameter ranging from about 0.2 mm to about 20 mm and a length ranging from about 10 mm to about 200 mm, depending on the particular application. In specific embodiments, flat splines are 3.5 mm to 5 mm, 5 mm to 10 mm, 10 mm to 15 mm, 15 mm to 20 mm in width, or any range therewithin (e.g., 3.5 mm to 10 mm); 3.5 mm to 5 mm, 5 mm to 10 mm. 10 mm to 15 mm, 15 mm to 20 mm in height, or any range therewithin (e.g., 3.5 mm to 10 mm); and 10 mm to 20 mm, 20 mm to 40 mm, 40 mm to 80 mm, 80 mm to 120 mm, 120 mm to 150 mm or 150 to 200 mm in length, or any range therewithin (e.g., 10 mm to 40 mm), or any permutation of the foregoing (e.g., a width of 5 mm to 10 mm, a height or 3.5 to 5 mm, and a length of 20 to 40 mm). In other embodiments, rounded splines are 3.5 mm to 5 mm, 5 mm to 10 mm, 10 mm to 15 mm, 15 mm to 20 mm in diameter, or any range therewithin (e.g., 3.5 mm to 10 mm) and 10 mm to 20 mm, 20 mm to 40 mm, 40 mm to 80 mm, 80 mm to 120 mm, 120 mm to 150 mm or 150 to 200 mm in length, or any range therewithin (e.g., 10 mm to 40 mm), or any permutation of the foregoing (e.g., a diameter of 5 mm to 10 mm and a length of 20 to 40 mm).

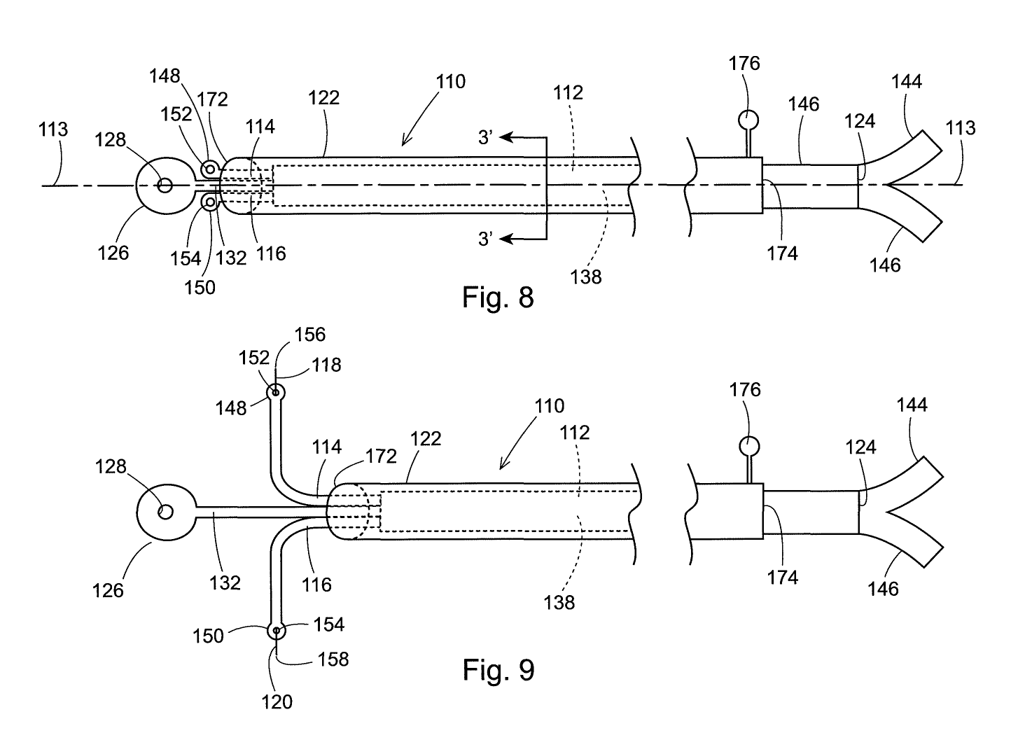

In a second specific embodiment, shown in FIG. 8, the device of the present invention is a fluid delivery catheter 110 comprising a central catheter component 112 having an elongate length with a longitudinal axis 113, one or more (and preferably two) flexible, resilient actuators that, in this specific embodiment, are formed as tissue penetrator presentation tubes 114, 116 that extend from the distal portion of the central catheter component 112. At least a portion of the tissue presentation tubes 114, 116 are movable between a constrained configuration which is oriented substantially parallel to the central longitudinal axis 113 of the catheter assembly and an unconstrained configuration which is oriented substantially non-parallel to the central longitudinal axis 113 of the catheter.

The catheter further comprises one or more (and preferably two) flexible, elongate tissue penetrators 118, 120 that extend through the two tissue penetrator presentation tubes 114, 116, and an exterior deployment tube 122 that extends over portions of the lengths of the central catheter component 112, the tissue penetrator presentation tubes 114, 116, and the middle rail 132.

The central catheter component 112 and the exterior deployment tube 122 may be constructed of any materials suitable for constructing catheters. Examples of such materials include, but are not limited to, silicone, polyurethane, nylon, Dacron, and PEBAX™.

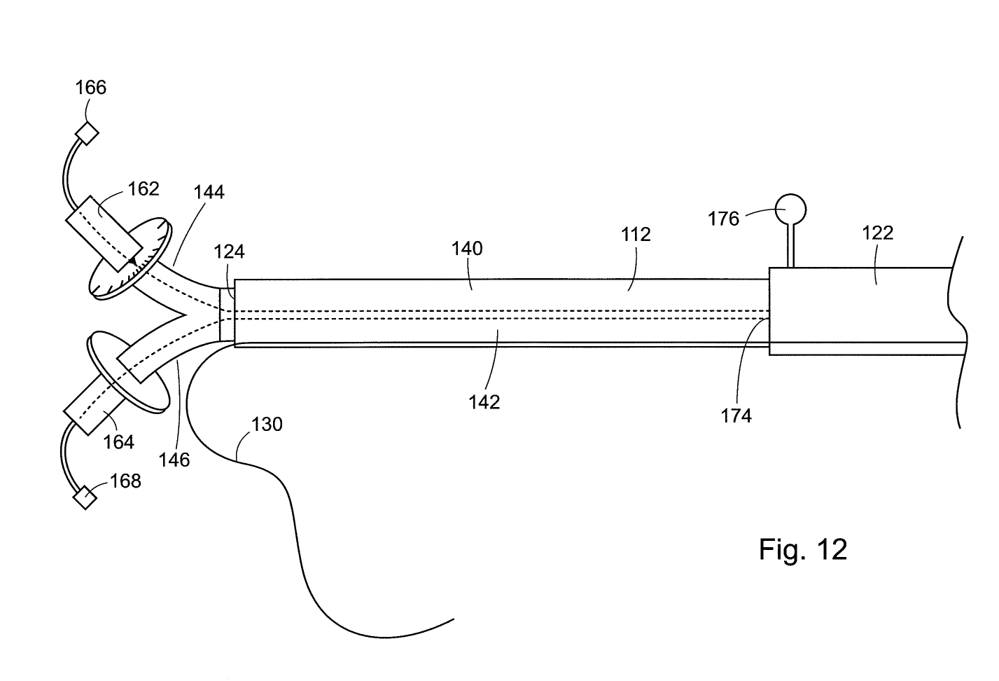

The tissue penetrators 118, 120 connect to respective hubs 166, 168 (FIG. 12). One or more of the pair of tissue penetrators 118, 120 preferably has a diameter of about 0.2 mm (33 gauge) to about 3.4 mm (10 gauge), more preferably 0.8 mm to 1.3 mm (about 18 to 21 gauge). One or more of the pair of tissue penetrators may have a standard bevel, a short bevel or a true short bevel. The pair of tissue penetrators 118, 120 are preferably constructed of materials that allow the tissue penetrators to flex along their lengths. Examples of such materials include, but are not limited to, nickel, aluminum, steel and alloys thereof. In a specific embodiment, the tissue penetrators are constructed of nitinol. The full length of the tissue penetrators 118, 120 can be constructed of a single material, or the distal ends (e.g., the distal 1 mm to the distal 20 mm), including the tips 156, 158, of the tissue penetrators 118, 120 may be constructed of one material and connected to the respective hubs 166, 168 via a tubing constructed of a different material, e.g., plastic.

One or more of the pair of tissue penetrator presentation tubes 114, 116 is preferably constructed of a flexible, resilient material. Such flexible, resilient material can be deformed, e.g., when the tissue penetrator presentation tubes 114, 116 are in the straight, constrained configuration of FIG. 8, but returns to its original shape when the deformation force is removed, e.g., when the tissue penetrator presentation tubes 114, 116 are in the curved, unconstrained configuration shown in FIG. 9. Any such flexible, resilient material can be used, including but not limited to surgical steel, aluminum, polypropylene, olefinic materials, polyurethane and other synthetic rubber or plastic materials. The pair of tissue penetrator presentation tubes 114, 116 is most preferably constructed of a shape memory material. Examples of such shape memory materials include, but are not limited to, copper-zinc-aluminum-nickel alloys, copper-aluminum-nickel alloys, and nickel-titanium (NiTi) alloys. In a preferred embodiment, the shape memory material is nitinol.

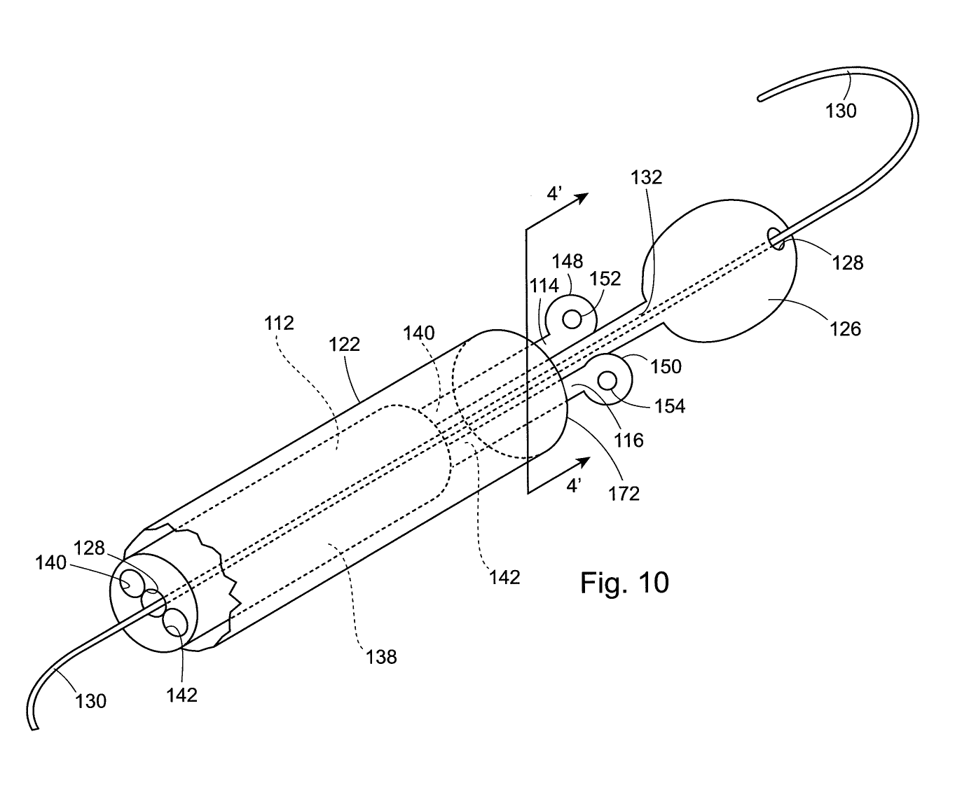

The central catheter component 112 has a flexible elongate length with opposite proximal 124 and distal 126 ends (FIG. 8). The distal end 126 of the central catheter component is formed as a guide tip that has an exterior shape configuration that will guide the distal end 126 through a biological space or conduit. A guide wire bore 128 within middle rail 132 extends through the center of the central catheter 112 from the proximal end 124 to the distal end 126. The guide wire bore 128 receives a flexible, elongate guide wire 130 for sliding movement of the bore 128 over the wire (FIG. 10). The guide wire 130 is used to guide the catheter assembly through a biological space or conduit. In certain embodiments, the guide wire 130 has a solid core, e.g., stainless steel or superelastic nitinol. The guide wire may optionally be constructed of radiopaque material, either in its entirety or at its distal portions (e.g., the most distal 1 mm to 25 mm or the most distal 1 mm to 3 mm). The guide wire 130 may optionally be coated with a medically inert coating such as TEFLON®. In other embodiments, the catheter assembly is a rapid-exchange catheter assembly wherein a guide wire is positioned on the distal end of the guide tip 126 and extends therefrom.

A narrow middle rail 132 surrounding the guide wire bore 128 extends from the guide tip of the catheter distal end 126 toward the catheter proximal end 124. The middle rail 132 connects the guide tip 126 to a base portion 138 of the central catheter component.

The central catheter component base portion 138 has a cylindrical exterior surface that extends along the entire length of the base portion. The base portion 138 extends along a majority of the overall length of the central catheter component 112. As shown in FIG. 10, the guide wire bore 128 extends through the center of the central catheter component base portion 138. In addition, a pair of tissue penetrator lumens 140, 142 also extend through the length of the central catheter component base portion 138 alongside the guide wire bore 128. At the proximal end 124 of the central catheter component, a pair of ports 144, 146 communicate the pair of lumens 140, 142 with the exterior of the central catheter component 112 (FIG. 8).

In an alternative embodiment, the medical device of FIG. 8 also may comprise a single flexible, resilient actuator that is formed as a tissue penetrator presentation tube, a single flexible, elongate tissue penetrator that extends through the tissue penetrator presentation tube and connects to a hub, and an exterior deployment tube that extends over portions of the lengths of the central catheter component, the tissue penetrator presentation tube, and the middle rail.

The pair of first and second tissue penetrator presentation tubes 114, 116 project from the catheter central component base portion 138 toward the catheter distal end 126. Each of the tissue penetrator presentation tubes is formed as a narrow, elongate tube having a proximal end that is secured to the central catheter component base portion 138, and an opposite distal end 148, 150. Each of the first and second tissue penetrator presentation tubes 114, 116 has an interior bore 152, 154 that communicates with the respective first tissue penetrator lumen 140 and second tissue penetrator lumen 142 in the central catheter component base portion 138.

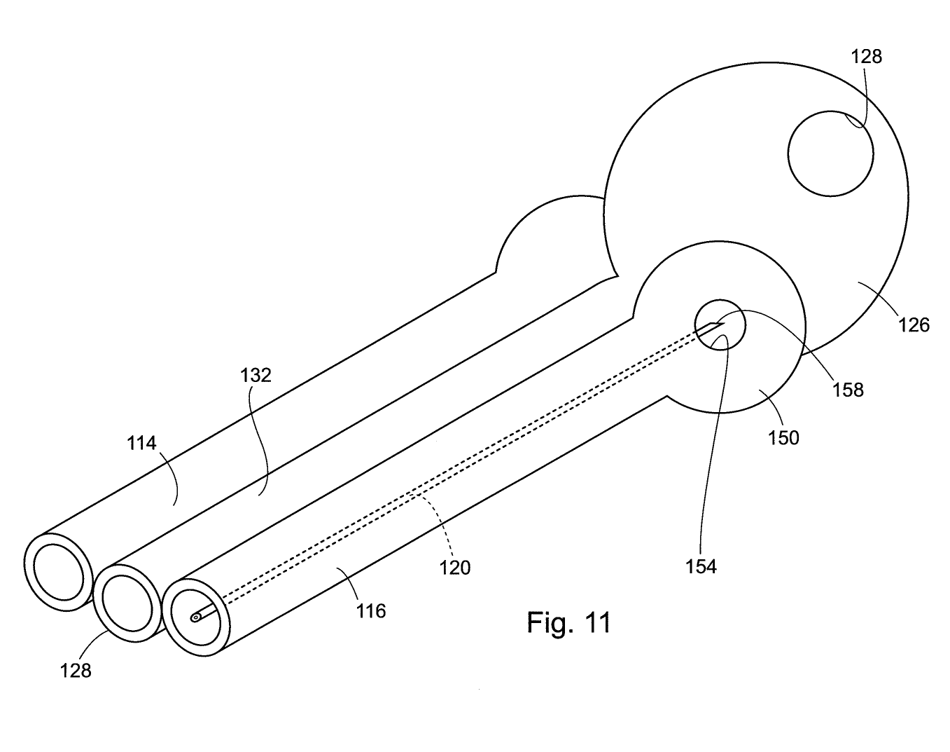

As shown in FIGS. 10 and 11, the exterior configurations of the tissue penetrator presentation tubes 114, 116 are matched to the middle rail 132 so that the lengths of the tissue penetrator presentation tubes 114, 116 may be positioned side-by-side on opposite sides of the middle rail 132. The tissue penetrator tube distal ends 148, 150 can be formed as guide tip surfaces that also facilitate the passage of the catheter through a vascular system. The tissue penetrator tube distal ends 148, 150 are preferably larger in diameter than the tissue penetrator presentation tubes 114, 116. In a specific embodiment, the tissue penetrator tube distal tips 148, 150 are rounded and bulbous tips. Such tips are atraumatic and the tubes will not inadvertently puncture the wall of a biological space or conduit. The tips 148, 150 are exposed and do not extend outwardly beyond the diameter of the guide tip 126.

Each of the tissue penetrator tubes 114, 116 is preferably constructed of a shape memory material, such as nitinol. The tubes 114, 116 are formed with curved, unconstrained configurations shown in FIG. 9. The tubes 114, 116 move to the curved, unconstrained configurations shown in FIG. 9 when no constraining force is applied against the tubes. In order for the presentation tubes 114, 116 to lie in straight, constrained configurations along the middle rail 132, a constraining force must be applied to the tubes to keep them in their straight, constrained positions shown in FIG. 8. As each of the tubes 114, 116 moves from its straight, constrained configuration shown in FIG. 9 to its curved, unconstrained configuration shown in FIG. 9, the tissue penetrator bores 152, 154 extending through the tubes also move from straight configurations to curved configurations.

The pair of tissue penetrators 118, 120, from their distal tips to the hubs 166, 168, have lengths that are slightly longer than the combined lengths of the tissue penetrator lumens 140, 142 extending through the central catheter base portion 138 and the tissue penetrator bores 152, 154 extending through the tissue penetrator presentation tubes 114, 116. The tips 156, 158 of the tissue penetrators 118, 120 are positioned adjacent to the distal ends 148, 150 of the tissue penetrator presentation tubes 114, 116 and are positioned inside of the bores 152, 154 of the tubes in the constrained configuration of FIG. 8. The opposite, proximal ends of the tissue penetrators 118, 120 project out through the side ports 144, 146 of the central catheter 112. The pair of tissue penetrators 118, 120 are dimensioned to easily slide through the tissue penetrator lumens 140, 142 of the central catheter component 112 and the tissue penetrator bores 152, 154 of the tissue penetrator presentation tubes 114, 116. The side ports 144, 146 of the central catheter component 112 are preferably at 20° to 90° angles to the central catheter proximal end 124, most preferably at 30° to 60° angles to the central catheter proximal end 124.

A pair of manual operator movement to linear movement controllers 162, 164 can be connected to the proximal ends of the tissue penetrators 118, 120 and can be secured to the central catheter ports 144, 146 (FIG. 12). The controllers 162, 164 can be constructed to convert operator movement into controlled linear movement of the tissue penetrators 118, 120 through the central catheter tissue penetrator lumens 140, 142 and through the tissue penetrator presentation tube bores 152, 154. In one embodiment, there are rotating controllers 162, 164 that can be manually moved in one direction, such that the tissue penetrator injection tips 156, 158 at the tissue penetrator distal ends can be adjustably positioned to extend a desired length out from the tissue penetrator tube bores 152, 154 at the tissue penetrator tube distal ends 148, 150. By rotating the controllers in the opposite direction, the tissue penetrators 118, 120 can be retracted back into the tissue penetrator tube bores 152, 154. Each of the operator movement to linear movement controllers 162, 164 can be provided with a hub 166, 168 that communicates with the interior bore extending through the tissue penetrators 118, 120 and can be used to connect a syringe or tubing containing a solution of a diagnostic or therapeutic agent.

The exterior deployment tube 122 has a tubular length that surrounds the central catheter 112, the tissue penetrator presentation tubes 114, 116, and the middle rail 132. The deployment tube 122 can be mounted on the central catheter component 112 and the pair of tissue penetrator presentation tubes 114, 116 for sliding movement to a forward position of the deployment tube 122 where an open distal end 172 of the deployment tube is positioned adjacent the distal ends 148, 150 of the tissue penetrator presentation tubes 114, 116 as shown in FIG. 8, and a rearward position of the deployment tube 122 where the tube distal end 172 is positioned adjacent to the connection of the tissue penetrator presentation tubes 114, 116 with the central catheter component 112 as shown in FIG. 9. The opposite proximal end 174 of the deployment tube 122 can be provided with a mechanical connection 176 to the central catheter 112. The mechanical connection 176 enables the deployment tube 122 to be moved between its forward and rearward positions relative to the central catheter 112 and the tissue penetrator presentation tubes 114, 116 (FIGS. 8 and 9). Such a connection could be provided by a thumbwheel, a sliding connection, a trigger or push button or some other connection that is manually operable to cause the deployment tube 122 to move relative to the central catheter 112 and the presentation tubes 114, 116. When the deployment tube 122 is moved to its forward position shown in FIG. 8, the tube distal end 172 passes over the lengths of the tissue penetrator presentation tubes 114, 116 and moves the presentation tubes to their constrained positions extending along the opposite sides of the central catheter middle rail 132. When the deployment tube 122 is moved to its rearward position shown in FIG. 9, the distal end 172 of the deployment tube is retracted from over the length of the tissue penetrator presentation tubes 114, 116 and gradually allows the presentation tubes 114, 116 to release their constrained energy and move to their curved, unconstrained configurations shown in FIG. 9.

In use of the catheter 110, the deployment tube 122 is in the forward position shown in FIG. 8. The guide wire 130 is positioned in a biological space or conduit (such as an artery or vein) in a known manner. The catheter is then advanced into the biological space or conduit over the guide wire. The guide wire 130 extends from the biological space or conduit, and enters the central catheter component distal end 126 through the guide wire lumen 128. The wire 130 passes through the length of the central catheter 112 and emerges at the proximal end of the central catheter component adjacent to the catheter ports 144, 146, where the guide wire 130 can be manually manipulated.

The catheter 110 can be advanced through the biological space or conduit and can be guided by the guide wire 130. Radiopaque markers may optionally be provided on the assembly to monitor the position of the assembly in the biological space or conduit. Any material that prevents passage of electromagnetic radiation is considered radiopaque and may be used. Useful radiopaque materials include, but are not limited to, platinum, gold, or silver. The radiopaque material can be coated on the surface of all or a part of the tip 126, on all or part of the presentation tubes 114, 116, on all or part of the tissue penetrators 118, 120, on the guide wire 130, or on any combination of the foregoing structures. Alternatively, a ring of radiopaque material can be attached to the tip 126. The assembly may optionally be provided with onboard imaging, such as intravascular ultrasound or optical coherence tomography. The tip of the assembly may optionally be provided with optics that are useful for determining the position of the assembly or the characteristics of the surrounding biological conduit. When the assembly is at a desired position, the exterior deployment tube 122 can be moved from its forward position shown in FIG. 8 toward its rearward position shown in FIG. 9 by manual manipulation of the mechanical connection 176.

As the deployment tube 122 is withdrawn from over the pair of tissue penetrator presentation tubes 114, 116, the constrained energy of the tissue penetrator presentation tubes 114, 116 is released and the tubes move toward their unconstrained, curved configurations shown in FIG. 9. This movement positions the tissue penetrator bores 152, 154 at the tissue penetrator tube distal ends 148, 150 against the interior surfaces of the biological space or conduit into which the assembly 110 has been inserted.

The operator movement to linear movement controllers 162, 164 then can be manually operated to extend the tissue penetrator distal ends 156, 158 from the tissue penetrator bores 152, 154 at the tissue penetrator presentation tube distal ends 148, 150. A gauge may be provided on each of the operator movement to linear movement controllers 162, 164 that provides a visual indication of the extent of the projection of the tissue penetrator tips 156, 158 from the tissue penetrator tube ends 148, 150 as the controllers 162, 164 are rotated. The controllers also could provide an audible sound or tactile feel such as clicking to indicate incremental distance steps of the tissue penetrator movements. This deploys the tissue penetrator tips 156, 158 a desired distance into the walls of the biological space or conduit.

In a third specific embodiment, a medical device of the instant invention is a fluid delivery catheter comprising one or more tissue penetrators constructed of a flexible, resilient material. In certain aspects, the medical device of the present invention has a central longitudinal axis, and comprises one or more tissue penetrators, wherein the one or more tissue penetrators can exist in a constrained configuration in which a length of said one or more tissue penetrators is oriented substantially parallel to the longitudinal axis of said medical device and an unconstrained configuration in which at least a portion of the length of said one or more tissue penetrators is oriented substantially non-parallel to the device's central longitudinal axis. After the device is positioned at a target site adjacent to the wall of a biological space or conduit, one or more tissue penetrators (and if desired, all of the tissue penetrators) may be released from a constrained configuration and permitted to adopt an unconstrained configuration, thereby making contact with the wall of the biological space or conduit. The one or more tissue penetrators may be of any shape, and in preferred embodiments, the movement of the one or more tissue penetrators from the constrained configuration to the unconstrained configuration occurs upon release of a constraining force by the device operator but without the input by the operator of any deforming forces to the device or the target tissue.

In a preferred embodiment, tissue penetrators are constructed of flexible, resilient material that is capable of being constrained upon the application of a constraining force, e.g., when the tissue penetrators are in the constrained configuration, and adopts its original unconstrained shape when the constraining force is removed, e.g., when the tissue penetrators are in the unconstrained configuration. Any such flexible, resilient material can be used, including but not limited to surgical steel, aluminum, polypropylene, olefinic materials, polyurethane and other synthetic rubber or plastic materials. The one or more tissue penetrators are most preferably constructed of a shape memory material. Examples of such shape memory materials include, but are not limited to, copper-zinc-aluminum-nickel alloys, copper-aluminum-nickel alloys, and nickel-titanium (NiTi) alloys. In a preferred embodiment, the shape memory material is nitinol. In a preferred embodiment, when the tissue penetrators assume the unconstrained configuration, the shape memory properties of the material from which each tissue penetrator is formed cause the tissue penetrators, without the application of any external deforming force, to move from a position substantially parallel to the longitudinal axis of the medical device to a position substantially perpendicular to the longitudinal axis of the medical device.

In a preferred embodiment, the tissue penetrators are maintained in the constrained configuration by an exterior catheter component having a tubular configuration that surrounds the tissue penetrators. A mechanical connection is provided between the exterior catheter component and the central catheter component to which the tissue penetrators are attached. The mechanical connection enables the exterior catheter component to be moved rearwardly along the length of the central catheter component, thereby uncovering the constrained one or more tissue penetrators and permitting the one or more tissue penetrators to assume an unconstrained configuration wherein they make contact with the target delivery site. One of ordinary skill in the art would appreciate that this specific embodiment may be readily adapted to incorporate radiopaque markers to facilitate positioning of the device or rapid-exchange features to facilitate the use of the device.

The medical device of the present invention, in its various embodiments, permits delivery of fluids into distinct layers of a vascular wall. The vascular wall consists of numerous structures and layers, structures and layers, including the endothelial layer and the basement membrane layer (collectively the intimal layer), the internal elastic lamina, the medial layer, and the adventitial layer. These layers are arranged such that the endothelium is exposed to the lumen of the vessel and the basement membrane, the intima, the internal elastic lamina, the media, and the adventitia are each successively layered over the endothelium as described in U.S. Pat. App. Publication No. 2006/0189941A1. With the medical devices of the present invention, the depth to which the tissue penetrator tips 156, 158 can penetrate into the target tissue can be controlled by rotating the controllers 162, 164. For example, if the target layer is the adventitial layer, the constrained energy of the tubes 114, 116 is released, the tubes adopt their unconstrained, curved configurations shown in FIG. 9, and the tissue penetrator tips 156, 158 are advanced with the controllers to a length sufficient for penetration to the depth of the adventitial layer. Likewise, if the target layer is the medial layer, the constrained energy of the tubes 114, 116 is released, the tubes adopt their unconstrained, curved configurations shown in FIG. 9, and the tissue penetrator tips 156, 158 are advanced with the controllers to a length sufficient for penetration to the depth of the medial layer.

With the tissue penetrators embedded in the desired layer of the wall of the biological space or conduit, a fluid can then be delivered through the tissue penetrators 118, 120. When the delivery of the fluid is complete, the controllers 162, 164 can be operated to withdraw the tissue penetrator tips 156, 158 back into the interior bores 152, 154 of the tissue penetrator presentation tubes 114, 116. The deployment tube 122 can then be moved to its forward position where the deployment tube distal end 172 moves the tissue penetrator presentation tubes 114, 116 back to their constrained positions shown in FIG. 8. When the deployment tube 122 has been moved to its full forward position shown in FIG. 8, the assembly can then be repositioned or withdrawn from the body.

The medical device of the instant invention also permits delivery of fluids to plaque deposits on the inside of the wall of the biological conduit or within the wall of the biological conduit.

The medical device of the instant invention also permits delivery of fluids to extracellular spaces or tissues located outside of the outer wall of the biological space or conduit (e.g., to the exterior surface of a blood vessel or to muscle positioned against the outer surface of vessel such as myocardium).

One advantageous feature of the devices of the present invention is that the actuators, by virtue of their design, make contact with less than the complete circumference of the inner wall of a biological conduit following their deployment therein. In preferred embodiments, the actuators make contact with less than 100% of the circumference of the inner wall of a biological conduit in which they are deployed. More preferably, the actuators make contact with less than 75%, 50% or 25% of the circumference of the inner wall of a biological conduit in which they are deployed. Most preferably, the actuators make contact with less than 10%, 5%, 2.5%, 1%, 0.5% or 0.1% of the circumference of the inner wall of a biological conduit in which they are deployed.

The devices can be used to deliver fluids comprising a variety of therapeutic and/or diagnostic agents to a wall of a biological space or conduit. Therapeutic agents include, but are not limited to proteins, chemicals, small molecules, cells and nucleic acids. A therapeutic agent delivered by the device may either comprise a microparticle or a nanoparticle, be complexed with a microparticle or a nanoparticle, or be bound to a microparticle or a nanoparticle. Protein agents include elastases, antiproliferative agents, and agents that inhibit vasospasm. The use of the devices for delivery of an elastase is specifically contemplated. Several published patent applications (WO 2001/21574; WO 2004/073504; and WO 2006/036804) teach that elastase, alone and in combination with other agents, is beneficial in the treatment of diseases of biological conduits, including obstruction of biological conduits and vasospasm. Diagnostic agents include, but are not limited to, contrast, microparticles, nanoparticles or other imaging agents.

A variety of distinct fluid delivery methods can be practiced with the device. In certain applications, distinct fluids can be delivered through each tissue penetrator of the device either simultaneously or sequentially. In other applications, the same fluid can be delivered through both tissue penetrators either simultaneously or sequentially. Embodiments and/or methods where a first fluid is delivered through both tissue penetrators followed by delivery of a second fluid through both tissue penetrators are also contemplated.

Methods of using the devices to deliver fluids into or through a wall of a biological space or conduit are also specifically contemplated. These methods comprise the steps of introducing the device into the biological space or conduit, advancing the device to a target site within the space or conduit, releasing the actuators from their constrained positions, optionally advancing the tissue penetrators through lumens in the actuators to penetrate to a desired depth into the wall of a biological space or conduit, delivering at least one fluid into or through the wall, optionally returning the tissue penetrators back into the lumens of the actuators, retracting the actuators to their constrained position, repositioning the device in the same or a different space or conduit for the delivery of additional fluid if so desired, and removing the device from the space or conduit. Also contemplated are methods of manufacturing the device.

Kits that comprise the device and at least one therapeutic agent or at least one a diagnostic agent, and combinations thereof are also specifically contemplated. A kit of the invention comprises, in one or more containers, a device of the instant invention and one or more of the therapeutic and/or diagnostic agents. In addition or in the alternative, the kits of the invention may provide an instructional material which describes performance of one or more methods of the invention, or a notice in the form prescribed by a governmental agency regulating the manufacture, use or sale of the device and the therapeutic and/or diagnostic agents, which notice reflects approval by the agency of manufacture, use or sale for human administration. In one embodiment, the therapeutic agent is an elastase, such as, but not limited to, pancreatic type I elastase, which is preferably human or porcine. In certain embodiments, the therapeutic agent may be pre-loaded into the medical device.

### EXAMPLE

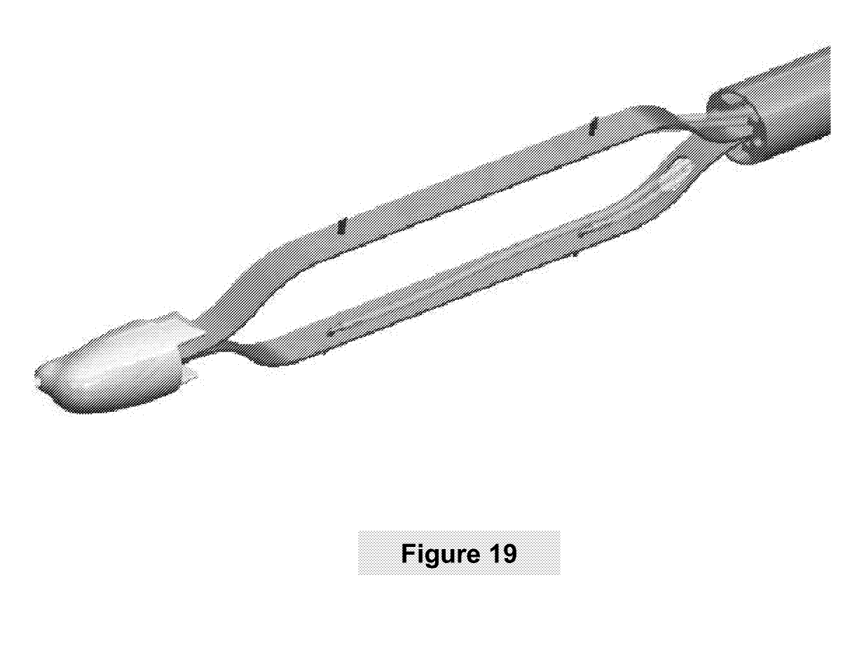

A prototype medical device of the embodiment depicted in FIG. 19 was constructed and operated.

The device has a central longitudinal axis, two splines made of a flexible metallic material from which project two tissue penetrators made of a more rigid metallic material. The splines are attached at their distal ends to a guide tip made of a plastic polymer. The construction was based on the following design principles. The construction was to include flat-wire springs to provide the outward expansion for the needles. A needle was to be connected at each end of each spring plateau so that there would be two opposing distal needles and two opposing proximal needles. The springs were intended to be constructed of a highly elastic metal such as Nitinol and would be set to a shape such that in the free state the springs are expanded. As used, the springs would be contained until the system in moved to the delivery location and the sheath withdrawn. As the sheath is withdrawn, the springs would expand and needles attached to the springs would be forced outward into the vessel wall.

The springs would be constructed with holes at each end of each spring plateau for needle connection. A needle would be connected at each hole so that there would be two opposing distal needles and two opposing proximal needles. Each needle would be constructed as an L-shaped tube that would pass through a hole in the spring. This attachment is designed to provide a secure and stable attachment. The conduit for drug delivery to the needles would pass through holes in the spring and attach to the inner ends of the needle tubing. This attachment method is designed to provide a junction that will be secure to the needles and easy to seal.

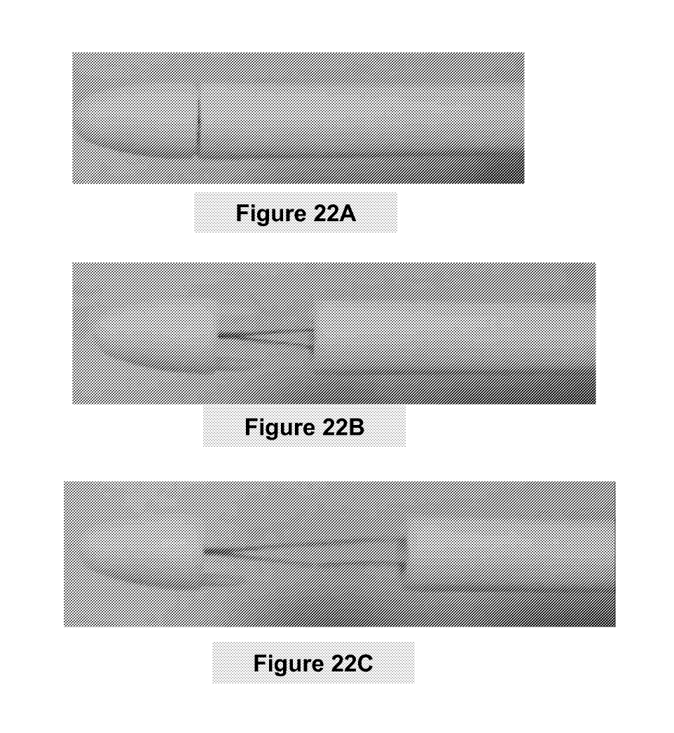

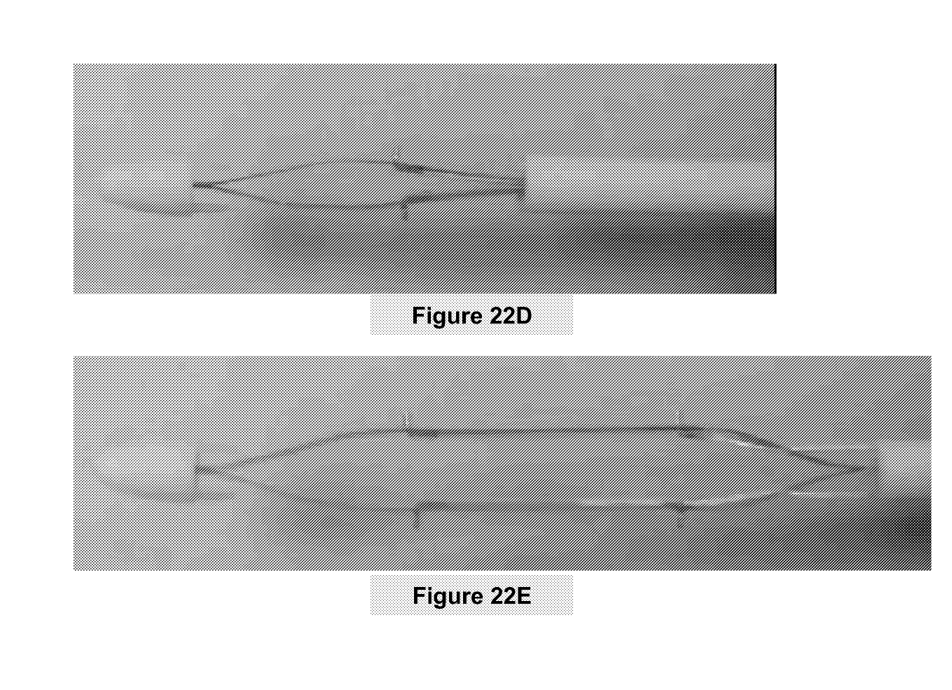

A 4-to-1 scale model, shown in FIG. 22, was built as a prototype of the design. The prototype was constructed using tempered steel for springs and hypodermic stainless steel tubing for the springs. The springs were formed into the expanded shape, heat set, and then tempered. The needle tubing was attached using adhesive. The drug delivery conduit was made of polymeric tubing that was attached to the ends of the needle tubing with adhesive. The polymer tip and sheath were made of stereolithography components.

In their constrained configuration, prior to deployment, the sheath is holds the springs, oriented substantially parallel to the longitudinal axis of the prototype device, in the compressed form (FIG. 22A). As the sheath is retracted (FIGS. 22B-22D), the springs move into an unconstrained configuration such that a portion of their lengths is oriented substantially non-parallel to the device's central longitudinal axis. In FIG. 22D, the first two opposing needles have exited the sheath. By the end of deployment, the springs have adopted an unconstrained configuration (FIG. 22E). Both pairs of opposing needles are exposed and the springs are fully expanded.

### Specific Embodiments, Citation of References

The present invention is not to be limited in scope by the specific embodiments described herein. The scope of the invention contemplated herein is not limited by the exemplary embodiments illustrated in the schematic drawings provided herein. Indeed, various modifications of the invention in addition to those described herein will become apparent to those skilled in the art from the foregoing description and accompanying figures, including medical devices other than catheters in which at least one component of the device displays the ability to adopt an unconstrained configuration following release from a constrained configuration, wherein the transition between the two configurations occurs upon release of a constraining force by the device operator but without the input by the operator of any deforming forces to the device or the target tissue. Such modifications are intended to fall within the scope of the appended claims.

Various references, including patent applications, patents, and scientific publications, are cited herein; the disclosure of each such reference is hereby incorporated herein by reference in its entirety.

1. A medical device for insertion into a biological conduit having a wall with an inner surface, said medical device having a longitudinal axis and comprising:
(a) at least one actuator having a constrained configuration in which said at least one actuator is oriented substantially parallel to the longitudinal axis of said medical device and an unconstrained configuration in which at least a portion of said at least one actuator is oriented substantially non-parallel to said longitudinal axis; and
(b) at least one tissue penetrator connected to said at least one actuator;
wherein said at least one actuator, upon removal of a constraining force, is released from said constrained configuration and adopts said unconstrained configuration.

2. The medical device of claim 1, wherein the adoption of said unconstrained configuration occurs without the application of any external deforming force to said at least one actuator.

3. The medical device of claim 2, wherein said unconstrained configuration has a predetermined shape.

4. The medical device of claim 3, wherein the penetrator is dimensioned to make contact with said inner surface when the actuator is insaid unconstrained configuration.

5. The medical device of claim 3, wherein the actuator is dimensioned to make contact with said inner surface when the actuator is in said unconstrained configuration.

6. The medical device of claim 4, wherein said contact comprises less than 100% of a circumference of said inner surface.

7. The medical device of claim 4, wherein said contact comprises less than 75% of a circumference of said inner surface.

8. The medical device of claim 4, wherein said contact comprises less than 50% of a circumference of said inner surface.

9. The medical device of claim 4, wherein said contact comprises less than 25% of a circumference of said inner surface.

10. The medical device of claim 4, wherein said contact comprises less than 10% of a circumference of said inner surface.

11. The medical device of claim 1, wherein said at least one actuator is constructed of a resilient material.

12. The medical device of claim 11, wherein said resilient material is selected from the group consisting of surgical steel, aluminum, polypropylene, olefinic materials, polyurethane, synthetic rubber, plastic, and a shape memory material.

13. The medical device of claim 12, wherein said at least one actuator is constructed of a shape memory material selected from the group consisting of a copper-zinc-aluminum-nickel alloy, a copper-aluminum-nickel alloy, and a nickel-titanium alloy.

14. The medical device of claim 13, wherein said at least one actuator is constructed of nitinol.

15. The medical device of claim 11, wherein the transition from said constrained configuration to said unconstrained configuration occurs at a rate that is dependent, after removal of said constraining force, upon the physical properties of the resilient material from which said at least one actuator is constructed.

16. The medical device of claim 1, wherein said at least one actuator is a pair of splines constructed of a resilient material, each spline of said pair of splines having an elongate length comprising opposite proximal and distal ends and an intermediate length therebetween, said spline proximal ends being secured together, said spline distal ends being secured together, and said intermediate lengths of said splines being capable of movement between said constrained configuration and said unconstrained configuration; said device further comprising:
(a) at least one tissue penetrator, said at least one tissue penetrator being positioned on at least one of said pair of splines, said at least one tissue penetrator having an interior bore that is adapted to permit delivery of a fluid from said interior bore; and,
(b) a track that is operatively connected to said pair of splines for movement between:
(i) a forward, extended position of said track along the lengths of said pair of splines where the operative connection of said track to said pair of splines constrains said pair of splines in said constrained configuration; and
(ii) a rearward, retracted position of said track from the length of said pair of splines where the operative connection of said track to said pair of splines allows said pair of splines to adopt said unconstrained configuration.

17. The medical device of claim 16, further comprising:
an exterior tubular component extending around said pair of splines, said component being connected to said track for movement of said component with said track to a forward, extended position where said component fully envelops said pair of splines and said at least one tissue penetrator, and a rearward, retracted position where said component does not fully envelop said pair of splines and said at least one tissue penetrator.

18. The medical device of claim 16, further comprising:
(a) a zipper rail that extends along the length of each spline of said pair of splines; and
(b) a fluid delivery conduit that extends along the length of each spline of said pair of splines.

19. The medical device of claim 16, further comprising:
(a) a guide tip secured to said distal ends of said splines and holding said spline distal ends in said constrained configuration, said guide tip having a guide wire bore extending through said guide tip and said guide tip having a smooth exterior surface;
(b) a central catheter component having:
(i) an elongate length with opposite proximal and distal ends, said central catheter component distal end being secured to said proximal ends of said pair of splines;
(ii) a pair of fluid delivery lumens that extend through the length of said central catheter component, wherein each of said pair of fluid delivery lumens communicates with one of said fluid delivery conduits;
(iii) a pair of Luer hubs at said proximal end of said central catheter component, each Luer hub of said pair of Luer hubs communicates with one of said pair of fluid delivery lumens; and
(iv) a guide wire lumen extending through the length of said central catheter component;
(c) a guide wire having an elongate length with opposite proximal and distal ends, said guide wire extending from its proximal end, through said guide wire lumen, along said pair of splines, and through said guide wire bore of said guide tip; and,
(d) an exterior catheter component having an elongate tubular length with opposite proximal and distal ends, said exterior catheter component being mounted over said central catheter and over said pair of splines for movement of said component along the lengths of said central catheter component and said pair of splines between:
(i) a forward, extended position of said exterior catheter component where said distal end of said exterior catheter component engages with said guide tip and wherein said exterior catheter component covers said pair of splines and said at least one tissue penetrator; and
(ii) a rearward, retracted position of said exterior catheter component where said distal end of said exterior catheter component is positioned around said central catheter component such that the said pair of splines and said at least one tissue penetrator are uncovered;
said exterior catheter component further comprising a zipper track located inside said exterior catheter component that extends along the length of said exterior catheter component, said zipper track engaging in a sliding engagement with said zipper rails of said pair of splines, and said zipper track sliding onto said zipper rails of said pair of splines and causing said pair of splines to translate from said unconstrained configuration to said constrained configuration in response to said exterior catheter component being moved to the forward, extended position, and said zipper track sliding off of said zipper rails of said pair of splines and causing said pair of splines to translate from said constrained configuration to said unconstrained configuration in response to said exterior catheter component being moved to the rearward, retracted position.

20. The medical device of claim 1, further comprising at least one radiopaque marker.

21. The medical device of claim 1, further comprising a manually operated controller operatively connected between said pair of splines and said track, said controller being operable to selectively cause movement of said track relative to said pair of splines between the forward, extended position of said track and the rearward, retracted position of said track in response to manual operation of said controller.

22. The medical device of claim 21, wherein said controller comprises a rotatable thumbwheel.

23. The medical device of claim 21, wherein said controller is operable to selectively move said track relative to said pair of splines in discrete distance steps in response to manual operation of said controller.

24. The medical device of claim 21, wherein said controller is operable to produce an audible indication of each distance step of movement of said track relative to said pair of splines.

25. The medical device of claim 21, wherein said controller is operable to produce a tactile indication of each distance step of movement of said track relative to said pair of splines.

26. The medical device of claim 1, wherein said at least one actuator is at least one tissue penetrator presentation tube, said at least one tissue penetrator presentation tube being constructed of a resilient material, said at least one tissue penetrator presentation tube having an elongate length with opposite proximal and distal ends and an interior bore extending through the length of said at least one tissue penetrator presentation tube, said at least one tissue penetrator presentation tube being capable of translating between said constrained configuration and said unconstrained configuration; said device further comprising:
(a) a tissue penetrator received in said interior bore of said at least one tissue penetrator presentation tube for sliding movement of said tissue penetrator through said interior bore, said tissue penetrator having a distal tip, said tissue penetrator being capable of translating between one or more deployed positions where said distal tip of said tissue penetrator projects from said interior bore of said at least one tissue penetrator presentation tube at said distal end of said at least one tissue penetrator presentation tube, and one or more retracted positions where said distal tip of said tissue penetrator is withdrawn into said interior bore of said at least one tissue penetrator presentation tube; and,
(b) a deployment tube mounted for movement over said at least one tissue penetrator presentation tube, said deployment tube capable of translating between one or more forward positions of said deployment tube wherein said deployment tube constrains said at least one tissue penetrator presentation tube in said constrained configuration and one or more rearward positions of said deployment tube where said deployment tube is retracted from said at least one tissue penetrator presentation tube and releases said at least one tissue penetrator presentation tube so that said at least one tissue penetrator presentation tube may translate to said unconstrained configuration.

27. The medical device of claim 26, wherein said distal end of said at least one tissue penetrator presentation tube has a smooth exterior surface configured to guide said at least one tissue penetrator presentation tube through said biological conduit; said device further comprising:
(a) a central catheter having an elongate length with opposite proximal and distal ends, said distal end of said central catheter being formed as a guide tip having a smooth exterior surface configured to guide said distal end of said central catheter through said biological conduit, a wire lumen extending through the length of said central catheter from said proximal end to said distal end, said central catheter having a narrow middle rail that extends from said distal end of said guide tip of said central catheter toward said proximal end of said central catheter, said middle rail having a length with opposite exterior surfaces that are configured to receive the length of said at least one tissue penetrator presentation tube when said at least one tissue penetrator presentation tube is in the constrained position, said central catheter having a base portion that extends from said middle rail to said proximal end of said central catheter, said base portion of said central catheter being secured to said proximal end of said at least one tissue penetrator presentation tube, and said central catheter having at least one tissue penetrator lumen that extends through said length of said central catheter and communicate with said interior bore of said at least one tissue penetrator presentation tube, said tissue penetrator extending through said length of said at least one tissue penetrator lumen in said central catheter and said interior bore of said at least one tissue penetrator presentation tube; and
(b) at least one operator movement to linear movement controller operatively connected with said proximal end of said central catheter and operatively connected to said tissue penetrator, said at least one operator movement to linear movement controller being operative to move said tissue penetrator in the opposite forward and rearward directions through said interior bore of said at least one tissue penetrator presentation tube and through said at least one tissue penetrator lumen of said central catheter base portion in response to operator movement of said at least one operator movement to linear movement controller in opposite directions, whereby moving said at least one operator movement to linear movement controller in one direction causes said distal end of said tissue penetrator to be adjustably extended a desired distance out of said distal end of said at least one tissue penetrator presentation tube, and movement of said at least one operator movement to linear movement controller in the opposite direction causes said distal end of said tissue penetrator to be retracted into said interior bore of said at least one tissue penetrator presentation tube.

28. The medical device of claim 27, wherein said at least one operator movement to linear movement controller has a rotatable thumbwheel.

29. The medical device of claim 27, wherein said at least one operator movement to linear movement controller is operable to selectively move said tissue penetrator in discrete distance steps in response to manual operation of said at least one operator movement to linear movement controller.

30. The medical device of claim 27, wherein said at least one operator movement to linear movement controller is operable to produce an audible indication of each distance step movement of said tissue penetrator.

31. The medical device of claim 27, wherein said at least one operator movement to linear movement controller is operable to produce a tactile indication of each distance step movement of said tissue penetrator.

32. The medical device of claim 26:
wherein said central catheter is coaxial with the longitudinal axis;
wherein said at least one tissue penetrator presentation tube is one of a plurality of tissue penetrator presentation tubes having elongate lengths with opposite proximal and distal ends and interior bores extending through the lengths of the plurality of tissue penetrator presentation tubes; and,
wherein said proximal ends of said plurality of tissue penetrator presentation tubes are symmetrically arranged around said central catheter axis.

33. The medical device of claim 32, wherein at least some of said plurality of tissue penetrator presentation tubes having different lengths.

34. The medical device of claim 26, further comprising at least one radiopaque marker.

35. The medical device of claim 26, wherein said at least one radiopaque marker is on said distal end of said at least one tissue penetrator presentation or tube.

36. A method for delivering a fluid into or through a wall of a biological conduit, said method comprising the steps of:
(a) introducing the medical device of claim 1 into the biological conduit;
(b) advancing the device to a target site within the biological conduit;
(c) releasing said at least one actuator from the constrained configuration; and
(d) delivering at least one fluid into or through a wall of said biological conduit;
thereby delivering a fluid into or through said wall of said biological conduit.

37. The method of claim 36, further comprising the steps of:
(a) returning said at least one actuator to the constrained configuration; and,
(b) removing said device from said biological conduit,
thereby delivering a fluid into or through said wall of said biological conduit.

38. The method of claim 36, wherein said biological conduit is a blood vessel.

39. The method of claim 38, wherein said fluid comprises an elastase.

40. A kit comprising:
(a) a medical device of claim 1; and
(b) at least one therapeutic agent or at least one diagnostic agent.

41. The kit of claim 40, wherein the therapeutic agent comprises an elastase.
